(19) United States
(12) Patent Application Publication
(10) Pub. No.: US 2024/0359034 A1
(43) Pub. Date: Oct. 31, 2024

(54) DEVICES, SYSTEMS, AND METHODS FOR COMBINING AND/OR DELIVERING INJECTABLE MATERIALS

(71) Applicants: BOSTON SCIENTIFIC SCIMED, INC., MAPLE GROVE, MN (US); BOSTON SCIENTIFIC MEDICAL DEVICE LIMITED, Galway (IE)

(72) Inventors: Nitesh Ghananil Baviskar, Kaylan West (IN); Benjamin Cleveland, Bellingham, MA (US); Sanjay Kumar Chandra, Pune (IN); Katie Knowles, Providence, RI (US); Rajivkumar Singh, Thane (IN); Marshall O'Hearn, Davenport, FL (US)

(73) Assignees: BOSTON SCIENTIFIC SCIMED, INC., MAPLE GROVE, MN (US); BOSTON SCIENTIFIC MEDICAL DEVICE LIMITED, Galway (IE)

(21) Appl. No.: 18/646,208

(22) Filed: Apr. 25, 2024

Related U.S. Application Data
(60) Provisional application No. 63/462,109, filed on Apr. 26, 2023.

(51) Int. Cl. A61N 5/10; A61J 1/20; A61M 5/19

(52) U.S. Cl. CPC A61N 5/10; A61J 1/201; A61J 1/2041; A61J 1/2048; A61J 1/2096; A61M 5/19; A61N 2005/1092

(57) ABSTRACT

Devices, systems, and methods for combining and/or delivering an injectable material to a patient. The injectable material may be formed of two or more reactive components delivered in separate chambers. Various features are provided to reduce human error in combining the reactive components in the appropriate sequence to form the injectable material, generally at (e.g., immediately prior to) the time of delivery to a patient. The system includes a sleeve lock which facilitates coupling of a device having a first chamber containing a first component with a vial having a second chamber containing a second component to combine the first and second components. The combined first and second components are contained within the first chamber for delivery to a patient via an injection system, which may be fluidly coupled with the first chamber with the assistance of the sleeve lock.

### CROSS-REFERENCE TO RELATED APPLICATIONS

This application claims the benefit of priority under 35 U.S.C. § 119 to U.S. Provisional Application No. 63/462,109, filed Apr. 26, 2023, the entire disclosure of which is hereby incorporated by reference herein for all purposes.

### FIELD

The present disclosure relates generally to the field of devices for delivering materials, such as injectable materials, to a patient, and associated systems and methods. More particularly, the present disclosure relates to devices for combining components of injectable materials, and associated systems and methods.

### BACKGROUND

Various forms of cancer and other medical conditions are treated by local application of radiation therapy. However, various risks may accompany radiation therapy. Since the conception of conformal radiotherapy, physicians have paid attention to the radiation dose delivered to the target and surrounding tissues. Investigators have been able to correlate side effects to the amount of tissue receiving a certain radiation dose. And yet, time, distance, and shielding affect the dose that is delivered. The less time an area is exposed to radiation, the less dose is delivered. The greater the distance from the radiation, the less dose is delivered. Filler materials may be injected into a treatment area to provide a shield to tissue surrounding the target of the radiation therapy. For instance, numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Various systems provide filler material to treatment sites to decrease the radiation dose to tissue surrounding radiation target sites (e.g., to shield the rectum during radiotherapy for prostate cancer). Such filler materials are often reactive, and therefore are generally combined/mixed immediately prior to or even during delivery to the patient. Various systems are known for combining/mixing (e.g., in vitro) filler materials injected into radiation treatment areas. However, most such systems include numerous subcomponents, are complex to assemble, and are susceptible to filler mixing errors prior to delivery within a patient at a treatment site. Various challenges posed by such mixing systems may result in errors and mishaps which lead unnecessarily increased procedure time, and increased procedure costs. Solutions to these and other issues presented by combining and delivering injectable materials would be welcome in the art.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an injectable material combining and/or delivery system includes a multi-chamber device, a vial adaptor, and injection system, and a sleeve lock. In some aspects, the multi-chamber device defines a first chamber and a second chamber therein. In some aspects, the vial adaptor defines a vial-receiving chamber. In some aspects, the injection system has a material delivery device configured to be fluidly coupled with the multi-chamber device to deliver, to a patient, a first component ejected from the first chamber into the injection system, and a second component ejected from the second chamber into the injection system. In some aspects, the sleeve lock is configured to facilitate engagement and disengagement of the vial adaptor and the multi-chamber device as well as engagement and disengagement of the multi-chamber device and the injection system. In some aspects, the sleeve lock is mounted on the multi-chamber device, and either of the vial adaptor or the injection system is axially and rotatably moved with respect to the sleeve lock to be engaged therewith or disengaged therefrom.

In some aspects, the sleeve lock remains mounted on the multi-chamber device upon disengagement of the vial adaptor from the sleeve lock.

In some aspects, the sleeve lock is rotatably mounted on the multi-chamber device.

In some aspects, the system further includes a fluid exchange device within the vial adaptor. In some aspects, the vial adaptor and the fluid exchange device are configured to facilitate alignment of a vial within the vial adaptor to establish fluid communication between the vial and the first chamber of the multi-chamber device. In some aspects, the system further includes a vial defining a third chamber therein and having a two-part cap comprising an outer cap and a stopper configured to seal a component within the third chamber. In some aspects, the fluid exchange device includes a needle and a fluid exchange spike supporting the needle and defining a fluid exchange lumen therethrough. In some aspects, the stopper of the two-part vial cap includes a wall section with reduced thickness to facilitate piercing therethrough by the needle of the fluid exchange device to facilitate passage of the fluid exchange spike through the stopper thereafter to fluidly communicate the first chamber and the third chamber. In some aspects, the vial adaptor includes vial-engaging elements configured to engage a portion of the outer cap of the vial.

In accordance with various principles of the present disclosure, the multi-chamber device has a first nozzle in fluid communication with the first chamber and a second nozzle in fluid communication with the second chamber; and the injection system defines a first nozzle port configured to be fluidly coupled with the first nozzle, a second nozzle port configured to be fluidly coupled with the second nozzle, and a Y-connector fluidly coupling the first nozzle port and the second nozzle port with the material delivery device. In some aspects, a mixer is provided within the injection system configured to mix a first component ejected into the injection system from the first chamber of the multi-chamber device with a second component ejected into the injection system from the first chamber of the multi-chamber device.

In some aspects, the system further includes an adaptor system having a sleeve lock configured to facilitate engagement and disengagement of the adaptor system and the injection system, the adaptor system having a single port configured to be fluidly coupled with a single-nozzle device, a first nozzle configured to be fluidly coupled with the first nozzle port of the injection system, and a second nozzle configured to be fluidly coupled with the second nozzle port of the injection system.

In accordance with various principles of the present disclosure, a multi-chamber system for combining components of an injectable material, includes a multi-chamber device, a vial adaptor, a sleeve lock, and a fluid exchange device. In some aspects, the multi-chamber device defines a first chamber and a second chamber therein. In some aspects, the vial adaptor defines a vial-receiving chamber. In some aspects, the sleeve lock is configured to facilitate engagement and disengagement of the vial adaptor and the multi-chamber device. In some aspects, the fluid exchange device is within the vial adaptor and is configured to fluidly communicate the first chamber of the multi-chamber device with a third chamber defined by a vial receivable within the vial-receiving chamber of the vial adaptor. In some aspects, the sleeve lock is mounted on the multi-chamber device, and the vial adaptor is axially and rotatably moved with respect to the sleeve lock to be engaged therewith or disengaged therefrom.

In some aspects, the system further includes a protective cap removably positioned within the vial-receiving chamber of the vial adaptor to shield the fluid exchange device.

In some aspects, the vial adaptor includes radially-inwardly directed vial-engaging elements configured to engage a portion of a vial cap.

In some aspects, the fluid exchange device includes a needle and a fluid exchange spike supporting the needle and defining a fluid exchange lumen therethrough. In some aspects, the multi-chamber device has a first nozzle in fluid communication with the first chamber. In some aspects, the fluid exchange lumen of the fluid exchange spike is in fluid communication with the first chamber of the multi-chamber device via a port defined in the vial adaptor in fluid communication with the first nozzle of the multi-chamber device.

In accordance with various principles of the present disclosure, a method, for combining and/or delivering an injectable material to a patient, includes one or more of the following: coupling and engaging a vial adaptor with a multi-chamber device, coupling a vial with the vial adaptor, fluidly communicating the multi-chamber device with the vial, ejecting material from the multi-chamber device into the vial, aspirate the material within the vial into the multi-chamber device, disengaging the vial adaptor, coupling the multi-chamber device with an injection system, ejecting materials from the multi-chamber device into the injection system, and delivering the materials from the multi-chamber device to a patient via the injection system.

In some aspects, the multi-chamber device and vial adaptor are coupled with each other with the assistance of a sleeve lock.

In some aspects, the vial adaptor defines a vial-receiving chamber, and the multi-chamber device has a first chamber containing a first component and a second chamber containing a second component.

In some aspects, the vial defines a third chamber containing a third component.

In some aspects, fluidly communicating the multi-chamber device with the vial includes fluidly communicating the first chamber within the multi-chamber device with the third chamber within the vial.

In some aspects, ejecting materials from the multi-chamber device includes ejecting the first component out from the first chamber and into the third chamber.

In some aspects, aspirating materials from the vial includes aspirating the first component and the third component into the first chamber.

In some aspects, disengaging the vial adaptor includes disengaging the vial adaptor, with the vial within the vial-receiving chamber, from the sleeve lock with the sleeve lock remaining engaged with the multi-chamber device.

In some aspects, coupling the multi-chamber device with an injection system includes coupling the multi-chamber device with the injection system with the assistance of the sleeve lock carried by the multi-chamber device.

In some aspects, ejecting materials from the multi-chamber device and into the injection system includes ejecting the first and third component out from the first chamber and into the injection system.

In some aspects, ejecting materials from the multi-chamber device and into the injection system includes ejecting the second component out from the second chamber and into the injection system.

In some aspects, delivering materials to a patient includes delivering the combined first component, second component, and third component to a patient via the injection system.

In some aspects, the method further includes mixing the first component and the third component inside the third chamber, such as defined within the vial.

In some aspects, the method further includes mixing materials ejected from the multi-chamber device within the injection system. In some aspects, a second component ejected from the multi-chamber device is mixed with a first component or a combined first and third component once the components have been injected into the injection system.

In some aspects, the method further includes shaking the vial with the materials therein to combine the materials therein.

In some aspects coupling the sleeve lock with either the vial adaptor or the injection system includes bringing the sleeve lock axially into engagement with one of the vial adaptor or the injection system, and then rotating the sleeve lock with respect to the one of the vial adaptor or the injection system to maintain axial engagement therebetween.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to facilitate injection into a patient. For purposes of clarity and simplicity, not every element is labeled in every figure, nor is every element of each embodiment shown where illustration is not necessary to allow those of ordinary skill in the art to understand the disclosure. Moreover, reference characters may indicate elements in some figures which are illustrated in other figures, and which, for the sake of brevity, are described only with reference to the other figures.

The detailed description will be better understood in conjunction with the accompanying drawings, wherein like reference characters represent like elements, as follows:

FIG. 1 illustrates a perspective view of an example of an embodiment of a system for combining and/or delivering injectable materials to a patient formed in accordance with various principles of the present disclosure.

FIG. 2 illustrates an exploded perspective view of an example of an embodiment of a multi-chamber system formed in accordance with various principles of the present disclosure.

FIG. 3A-FIG. 3H illustrate various stages of preparing an injectable material, in accordance with various principles of the present disclosure, with an example of an embodiment f a multi-chamber system such as illustrated in FIG. 2.

FIG. 4A illustrates a partial distal perspective view of a multi-chamber system such as illustrated in FIG. 3D.

FIG. 4B illustrates a cross-sectional view of the multi-chamber system of FIG. 4A along line IVB-IVB.

FIG. 5 illustrates examples of embodiments of a multi-chamber device and an injection system, such as of a combining and/or delivery system formed in accordance with various principles of the present disclosure, fluidly coupled to deliver an injectable material to a patient.

FIG. 6A illustrates a partial distal perspective view of a multi-chamber system and injection system such as illustrated in FIG. 5.

FIG. 6B illustrates a partial cross-sectional view, along line VIB-VIB of FIG. 6A.

FIG. 7 illustrates an exploded perspective view of an example of an embodiment of an injection system and an example of an embodiment of an adaptor system, each formed in accordance with various principles of the present disclosure for engagement with the other.

FIG. 8A-FIG. 8E illustrate various stages of connecting a pre-treatment device with an injection system such as illustrated in FIG. 6A, delivering (e.g., injecting) a pre-treatment material (e.g., into a patient), and detaching the pre-treatment device after delivery of the pre-treatment material to the patient.

FIG. 9A-FIG. 9F illustrate perspective views of various examples of embodiments of adaptors which may be used with an injection system, and/or a combining and/or delivery system formed in accordance with various principles of the present disclosure.

FIG. 9G illustrates a cross-sectional view along line IXG-IXG of FIG. 9F.

FIG. 9H illustrates a detail cross-sectional view along line IXH-IXH of FIG. 9G.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical procedures involve delivery (e.g., injection) of injectable material(s) into the body before, during, or after the procedure. Preferably, the injectable material is biocompatible, and optionally biodegradable. The injectable material may serve a variety of purposes, including, without limitation, differentiating tissue (e.g., by creating a “bleb” or other raised or swelled region to distinguish an anatomical region), spacing anatomical structures from one another, otherwise affecting (e.g., shielding, coating, covering, modifying, etc.) an anatomical structure, etc. It will be appreciated that the term “tissue” is a broad term that encompasses a portion of or site within a body: for example, a group of cells, a group of cells and interstitial matter, an organ, a portion of an organ, an anatomical portion of a body, e.g., a rectum, ovary, prostate, nerve, cartilage, bone, brain, or portion thereof, etc. Moreover, reference may be made, herein, to a “target” in referring to an area of the patient's body at which a procedure is to be performed. However, it will be appreciated that such reference is to be broadly understood and is not intended to be limited to tissue or to particular procedures. Finally, reference may be made to target tissue, target location, target site, target tissue site, anatomical site, delivery site, deployment site, injection site, treatment site, etc., including combinations thereof and other grammatical forms thereof, interchangeably and without intent to limit.

Certain specific aspects of the present disclosure relate to placing an injectable material between target tissue to be treated and other tissues. For the sake of convenience, and without intent to limit, reference is made to an injectable material, such as a filler, including, without limitation, a gel composition. The injectable material may be delivered within the patient to displace the tissue relative to a tissue that is to be treated by a therapeutic procedure or otherwise (e.g., not necessarily therapeutic). Certain aspects of the present disclosure include displacing and/or shielding a tissue to protect the tissue against possible side effects of treatment of a target tissue, such as the effects of a treatment involving radiation or cryotherapy. In some aspects, the injectable material may displace anatomical tissue and/or may increase the distance between the target tissue and other tissues. For instance, if the target tissue is to be irradiated, the injectable material may space other tissues from the target tissue so that the other tissues are exposed to less radiation and/or are shielded from the radiation. In some aspects, the injectable material is injected as a filler in a space between tissues. A first tissue may then be treated by radiation, while the injectable material reduces passage of radiation therethrough into a second tissue. The first tissue may be irradiated while the second tissue, spaced by the injectable material, receives less radiation than it would have in the absence of the injectable material. An effective amount of an injectable material may be injected into a space between a first tissue to be treated and a second tissue which can be a critically sensitive organ. For instance, in the context of treatment of prostate cancer, an injectable material may be injected into the Denonvilliers' space (a region between the rectum and prostate) to create additional space between the rectum and prostate and/or to shield the rectum during treatment, thereby reducing rectal radiation dose and associated side effects.

In some aspects of the present disclosure, components of an injectable material are combined by a system formed in accordance with various principles of the present disclosure, and injected into or near a target site. It will be appreciated that terms such as combine, mix, blend, etc., (including other grammatical forms thereof) may be used interchangeably herein without intent to limit unless otherwise indicated. Reference is accordingly made herein to a combining system generically, without intent to specifically require active combining/mixing.

In accordance with various principles of the present disclosure, the injectable material may be a filler such as a hydrophilic polymer, a gel, a hydrogel, etc. For instance, the injectable material can include polymeric materials which are capable of forming a hydrogel upon crosslinking. Optionally, the polymer forms a hydrogel within the body. A hydrogel is defined as a substance formed when a polymer (natural or synthetic) is crosslinked via covalent, ionic, or hydrogen bonds to create a three-dimensional structure which entraps water molecules to form a gel. Naturally-occurring and synthetic hydrogel-forming polymers, polymer mixtures, and copolymers may be utilized as hydrogel precursors. In some aspects, the hydrogel can be formed by a composition formed by two or more constituents/components (e.g., mixing accelerant fluid, diluent, and polyethylene glycol (PEG) together), and may include one or more polysaccharide compounds or a salt thereof. For example, the composition may include a cellulose compound such as carboxymethyl cellulose (CMC) or salt thereof (e.g., CMC sodium), xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide.

In some aspects of the present disclosure, two or more components of an injectable material are provided separately, and are combined by a device, system, and method in accordance with various principles of the present disclosure to form a compound material to be injected into or near a target site by devices, systems, and methods in accordance with various principles of the present disclosure. The injectable material may be delivered within the patient to displace the tissue relative to a tissue that is to be treated by a therapeutic procedure or otherwise (e.g., not necessarily therapeutic). A composition to be injected into a patient may be a combination of two or more components combined by a device, system, or method formed in accordance with various principles of the present disclosure. For instance, devices, systems, or methods of the present disclosure may be used to combine a first component and a second component for injection into a patient. The first component may be a precursor, e.g., a first component to be combined with a second component to form the injectable compound. The second component may be an accelerator, an accelerant, an activating agent, a cross-linking inducing agent, a catalyst, an initiator, etc., which, upon combination with the precursor, produces the injectable compound, such as by altering the chemical composition or structure of the first component. The components may be combined prior to (e.g., immediately prior to or during) delivery (e.g., during injection) to the patient so that the injectable material does not have time to form into a structure which may be difficult to inject or otherwise deliver to the patient. As such, the combination of the first component and the second component may be such that the injectable compound attains its final desired properties/reaches its final form in situ.

In some embodiments, the injectable material is formed of a first component, a second component, and a third component. For instance, for various reasons it may be desirable to provide a first, precursor component in a solid form (e.g., to allow mixing at the time of delivery, and/or to be more stable for storage and/or transport). The first component is combinable with the third component, and the thus-formed combined composition (which may be referenced as the precursor) is then combinable with the second component once the medical professional is ready to deliver (e.g., inject) the injectable material to the patient. The second component may facilitate a crosslinking interaction between the first and third components, for example, by initiating or accelerating the crosslinking interaction of the first and third components. Typically, one or more of the components of the injectable materials are biocompatible polymers. In some aspects, one of the first component or third component is a reactive polymer, such as a cross-linkable and/or hydrophilic polymer component (e.g., PEG), and the other of the first component or third component is a diluent (e.g., mostly water) in which a solid or semi-solid form of the one of the first component or third component is dissolved or dispersed, and/or with which the one of the first component or third component is cross-linked (or at least cross-linkable, such as upon further combination with the second component), to form a precursor. The second component may be an accelerator, an accelerant, an activating agent, a catalyst, an initiator, etc. (such terms being used interchangeably herein without intent to limit), combinable and reactive with the precursor (formed from the first component and the third component) to form the desired injectable material. In one example of an embodiment, a first component, in the form of a cross-linking agent (specifically, trilysine, which contains multiple nucleophilic groups, specifically, amino groups), is mixed with a third component, in the form of a reactive polymer (specifically, PEG) that has been derivatized with reactive, optionally electrophilic, groups (specifically, succinimide ester groups), under acidic pH conditions where the succinimide ester groups and the amino groups do not react to any significant degree. When this mixture is combined with a second component, in the form of an accelerant (specifically, a basic buffer solution), the pH of the resulting mixture becomes basic, at which point the amino groups of the trilysine react with the succinimide ester groups of the PEG to form covalent bonds, thereby crosslinking the PEG and forming a hydrogel.

It will be appreciated that reference to “first”, “second”, or “third” is not intended to connote a particular nature of the material or the order in which the material is combined. As such, “first”, “second”, and “third” may be used to reference any of three components forming an injectable material in accordance with various principles of the present disclosure. A non-limiting example of such components combinable by devices, systems, or methods in accordance with various principles of the present disclosure includes a first component such as a reactive component, a solute, etc.; a second component such as a diluent with which the reactive component is to be combined to form a precursor; and a third component such as an accelerator combinable with the precursor to form an injectable material. The injectable material is a biocompatible material, such as a polymeric material, such as a filler, or such as a hydrogel.

In some examples, the composition may be or include a gel with a desired gel strength and/or viscosity, such as a biocompatible gel suitable for injection (e.g., through a needle), as discussed in further detail below. In one example of an embodiment, one of the components is a biocompatible polymeric component. More particularly, in one example of an embodiment, one of the components is a hydrophilic polymer, which may be natural or synthetic in origin, and may be anionic, cationic, zwitterionic, or neutrally charged. Non-limiting examples of hydrophilic polymers include natural hydrophilic polymers including proteins such as collagen and polysaccharides such as gellan gum, xanthan gum, gum arabic, guar gum, locust bean gum, alginate, and carrageenans, and synthetic hydrophilic polymers such as polyethylene glycols (PEG), PEG-methacrylates, PEG-methylmethacrylates, polyvinyl alcohols, polyacrylates and polymethacrylates, polyacrylic acids and their salts, polymethacrylic acids and their salts, polymethylmethacrylates, carboxymethylcelluloses, hydroxyethylcelluloses, polyvinylpyrrolidones, polyacrylamides such as N,N-methylene-bis-acrylamides or tris(hydroxymethyl) methacrylamides. The hydrophilic polymer may be modified to provide functional groups that are reactive with functional groups of a suitable cross-linking agent, which may be a covalent or ionic cross-linking agent.

The concentrations of gelling agent(s) in a composition formed in accordance with various principles of the present disclosure maybe at least about 0.01% by weight with respect to the total weight of the composition, and at most about 2.0% by weight with respect to the total weight of the composition, including increments of about 0.01% therebetween. For instance, the concentration of gelling agent(s) may range from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, a composition formed in accordance with various principles of the present disclosure may have a viscosity of at least about 0.001 pascal-second (Pa·s), and at most about 0.100 Pa·s at a shear rate of 130 s⁻¹. For instance, the composition may have a viscosity ranging from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, a composition formed in accordance with various principles of the present disclosure may have a viscosity of at least about 0.001 Pa·s, and at most about 0.050 Pa·s at a shear rate of 768 s⁻¹. For instance, the composition may have a viscosity ranging from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The various components forming the injectable material may be kept separate until the time of the procedure in which the final composition is used for various reasons, such as to maintain stability of the individual components. In some embodiments, a multi-chamber system includes separate chambers for components to be combined to form the injectable material to be delivered to the patient by an injection system. In some embodiments, a first component and a second component are separately contained within a first chamber and a second chamber, respectively, of a multi-chamber device. A third component may be contained in a separate device defining a third chamber of the multi-chamber system. To deliver the injectable material, the components of the first and third chambers are combined within the first chamber (e.g., to form a precursor), and then the components of the first and second chambers and injected together into the patient. The multi-chamber device may or may not mix the contents of the first chamber with the contents of the second chamber. For instance, the multi-chamber device may deliver and inject the components of the injectable material to an injection system, with the injection system including a mixer component configured to mix the components from the first chamber of the multi-chamber device with the contents from the second chamber of the multi-chamber device as those contents are injected from the multi-chamber device and the injection system into the patient. The already-combined first and third components are combined with the second component to form the desired form, structure, composition, properties, etc., of the injectable material to be delivered to and/or deposited within the patient. The final form, structure, composition, properties, etc., of the injectable material may be attained once the combined components are within the patient.

The present disclosure provides devices, systems, and methods for combining components to form an injectable composition, and corresponding medical devices, systems, and methods for use thereof and/or delivery to a treatment site of a patient. According to some aspects of the present disclosure, such as described above, a multi-chamber system may include a plurality of chambers for the one or more components of the injectable material and for combinations of such components. It will be appreciated that terms such as chamber, reservoir, container, vial, lumen, etc., may be used interchangeably herein without intent to limit, to refer to elements which contain, convey, hold, transport, collect, etc., a component (fluid, particulate, liquid, solid, gas, etc.) of an injectable material. Suitable chambers may include, for example, vials, syringes (e.g., a syringe barrel compatible with a manual or automatic injection system) and other fluid containers, such as configured for use with a suitable injection system. Examples of materials suitable for chambers of devices or systems of the present disclosure include, but are not limited to, cyclic olefin polymer, polypropylene, polycarbonate, polyvinyl chloride, and glass. In some aspects, one of these materials (e.g., cyclic olefin copolymer specifically) can have a coating applied to it (such as SiO₂ coating), which is advantageous so the coating can provide a primary oxygen barrier, behave as a glass-like layer, and/or can be applied using a vapor deposition process.

A combining device or system formed in accordance with various principles of the present disclosure to combine two or more components to form an injectable material may include and/or be removably connected to one or more injection systems which are configured to deliver the injectable materials to a patient. According to some aspects of the present disclosure, the filler compositions which may be used with various systems disclosed herein, e.g., the compositions prepared by the various devices, systems, methods disclosed herein, may have sufficient strength, e.g., gel strength, to withstand the forces on the continuity of the three-dimensional configuration (e.g., gel network) of the composition, and thereby minimize the effects of such forces. In the meantime, compositions with sufficient strength to withstand forces thereon may have a viscosity suitable for injection, e.g., a viscosity that does not cause the composition to become stuck in the reservoir(s), delivery lumen, needle, or other structure in which the composition is contained or through which it passes. According to some aspects of the present disclosure, the composition may maintain its three-dimensional structure until the composition is injected into a patient (e.g., through a needle), whereupon the structure may form fragments of the original continuous, three-dimensional network. Those fragments may have a diameter corresponding to the diameter of the lumen through which it passes into the patient (e.g., the lumen of an injection needle), such that the fragments are as large as possible in-vivo to retain as much of the three-dimensional structure of the composition as possible. Injection of these larger-sized particles or fragments is believed to increase the amount of time the gel remains within the tissue.

In some examples, the injection system includes a needle. In some embodiments, the needle may be a hypodermic needle, and may range from a size of 7-gauge (4.57 mm outer diameter (OD), 3.81 mm inner diameter (ID)) to 33-gauge (0.18 mm OD, 0.08 mm ID), e.g., a size of 16-gauge (1.65 mm OD, 1.19 mm ID), 18-gauge, 21-gauge (0.82 mm OD, 0.51 mm ID), 22-gauge (0.72 mm OD, 0.41 mm ID), 23-gauge (0.64 mm OD, 0.33 mm ID), or 24-gauge (0.57 mm OD, 0.31 mm ID). According to some aspects of the present disclosure, the size of the needle may be chosen based on the viscosity and/or components of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein. Examples of materials which may be used to form the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

As noted above, compositions used with systems disclosed herein may have large particulate matter (relative to the injection system lumen) and/or a high viscosity for passage through a lumen sized to inject the material into the patient. The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lb·f to about 25 lb·f, such as from about 10 lb·f to about 20 lb·f, e.g., about 15 lb·f. The loads measured for a given gel concentration may vary for different needles and flow rates.

According to some aspects of the present disclosure, a combining device or system can be included in a kit for introducing an injectable material into a patient, whereby the injectable material can include any of a variety of suitable compositions. Kits or systems may be configured to store one or more of the components of a composition until the medical professional is ready to mix the composition for delivery to a patient. For instance, compositions, such as hydrogels, may be prepared so that the precursor(s) and any related activating agent(s) are stored in the kit with diluents as may be needed. Applicators may be used in combination with the same. Kits formed in accordance with various principles of the present disclosure can be manufactured using medically acceptable conditions and contain components that have sterility, purity, and preparation that is pharmaceutically acceptable. Solvents/solutions may be provided in the kit or separately.

The kit may include one or more syringes and/or needles for mixing and/or delivery of the injectable material, and/or for additional aspects of the procedure in which the injectable material is to be used. The kit or system may comprise various components as set forth herein. For instance, a target site into which an injectable material is to be delivered may be pre-treated using one or more components of the kit. One example of a pretreatment includes hydrodissection, such as with saline, to create space for injectable material to be injected at or in the vicinity of the target tissue site. Once saline has been injected to the treatment site, a combining device or system can be connected to a needle (e.g., an 18-gauge spinal needle) to then deliver the injectable material to the treatment site. For instance, in treating prostate cancer, a 5-10 mm layer of filler (e.g., gel composition) may be injected along the posterior wall of the prostate between the prostate and rectum. Once the filler has been injected into the space between the rectum and prostate, ultrasound images can be obtained.

In accordance with various principles of the present disclosure, a combining and/or delivery system is configured to facilitate combining of components of an injectable material. In some aspects, the injectable material is a combination of a first component, a second component, and a third component, such as described above. A combining and/or delivery system formed in accordance with various principles of the present disclosure facilitates combining/mixing of the various components of an injectable material. Additionally or alternatively, a combining and/or delivery system formed in accordance with various principles of the present disclosure facilitates delivery of the injectable material to an injection system configured to deliver to and/or deposit the injectable material into (e.g., inject) a patient (e.g., to a target site within the patient). More particularly, various aspects of the present disclosure simplify assembly, alignment, mixing, dispensing, etc., of an injectable material which is combined or blended or mixed from separate components before delivery to a patient.

In some aspects, a combining and/or delivery system formed in accordance with various principles of the present disclosure includes a multi-chamber system with three distinct chambers for respective first, second, and third components of an injectable material to be delivered and injected into a patient. The present disclosure facilitates combination of the separately-provided components before a procedure, such as to form a precursor, as well as combination of the recently-combined components/precursor with another of the components of the injectable material for injection into the patient.

An example of an embodiment of a multi-chamber system disclosed herein is configured to facilitate the combining of the first, second, and third components of an injectable material before delivery to a patient. As described above, it may be desirable to provide the first, second, and third components separate from one another for combination only once a procedure is to be performed utilizing the injectable material formed by combining the first, second, and third components. The multi-chamber system may then be fluidly coupled with an injection system, formed in accordance with various principles of the present disclosure, to deliver the injectable material to a patient.

In an example of an embodiment disclosed herein, a multi-chamber system formed in accordance with various principles of the present disclosure includes a multi-chamber device having a barrel defining first and second separate chambers, and a vial defining a third chamber. The multi-chamber system further includes a vial adaptor configured to facilitate fluid coupling of the vial with the barrel to allow combining of a component within one of the two chambers of the barrel with a component within the vial. The vial and vial adaptor optionally are configured to facilitate a secure and/or properly-aligned fit of the vial within the vial adaptor. The multi-chamber system optionally includes a sleeve lock configured to facilitate secure manual coupling/engagement of the barrel of the multi-chamber device with the vial adaptor. Additionally or alternatively, the sleeve lock is configured to facilitate manual decoupling/disengagement of the barrel of the multi-chamber device from the vial adaptor. It will be appreciated that reference herein to facilitating manual coupling, engaging, decoupling, disengaging, etc. (including other grammatical forms thereof) is to be understood as being configured to allow ready, easy, simplified movements and/or steps without the need for additional tools or complex or difficult or time-consuming steps which may otherwise interfere with expeditious use of the devices and/or systems of the present disclosure, as may be appreciated by those of ordinary skill in the art.

A combining and/or delivery system formed in accordance with various principles of the present disclosure optionally further includes an injection system configured to deliver an injectable material to a patient (e.g., to inject the injectable material into the patient). The combining and/or delivery system optionally includes a sleeve lock configured to facilitate secure coupling/engagement of the barrel of the multi-chamber device with the injection system. Additionally or alternatively, the sleeve lock is configured to facilitate decoupling/disengagement of the barrel of the multi-chamber device from the injection system. The sleeve lock of the multi-chamber system may be used with both the vial adaptor as well the injection system.

The combining and/or delivery system optionally further includes an adaptor system configured to facilitate secure coupling/engagement of a pretreatment system with the injection system. Additionally or alternatively, the adaptor system is configured to facilitate decoupling/disengagement of the pretreatment system from the injection system. The pretreatment system is configured to deliver pretreatment materials to a patient via the injection system.

Various embodiments of combining and/or delivery devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified by common reference characters and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Finally, certain features in one embodiment may be used across different embodiments and are not necessarily individually labeled when appearing in different embodiments.

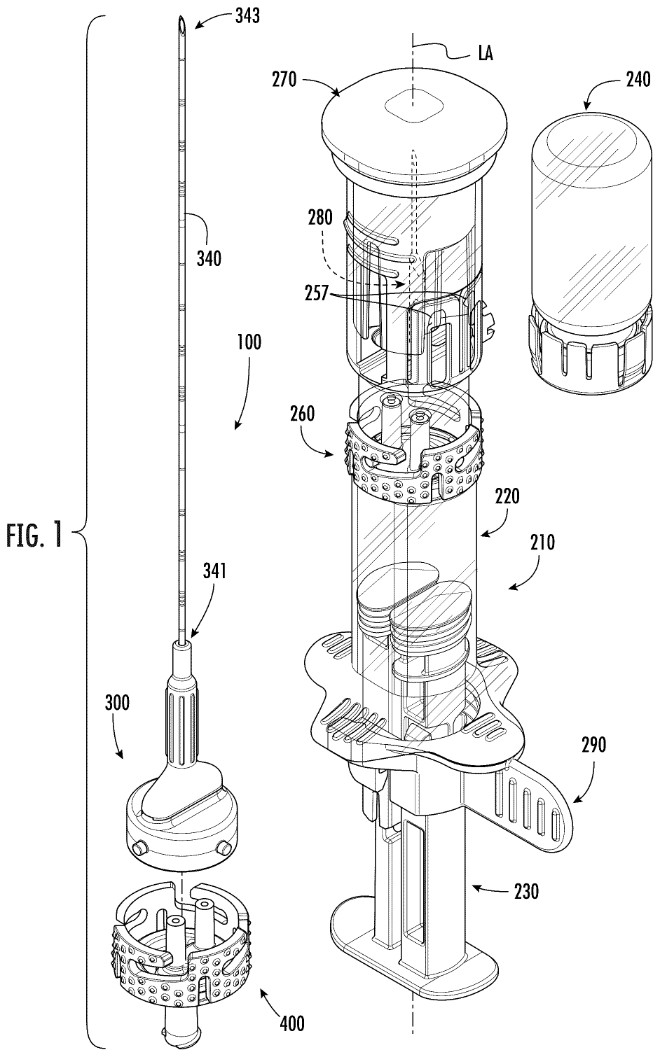

Turning now to the drawings, an example of an embodiment of a combining and/or delivery system 100 formed in accordance with various principles of the present disclosure is illustrated in FIG. 1. The combining and/or delivery system 100 includes a multi-chamber system 200 and an injection system 300. The multi-chamber system 200 may be used to transport components of an injectable material to a facility/location at which the injectable material is to be delivered into a patient. For the sake of convenience, and without intent to limit, reference may be made to delivery by injection into the patient (e.g., the injectable material is delivered to the patient by being injected into the patient), although the present disclosure need not be so limited. The material combined and/or delivered by a combining and/or delivery system 100 of the present disclosure may be any other type of material to be administered to a patient, such as other than by injection. The injectable material may, as described above, include two or more components which are to remain separate until the time of the procedure. Once a medical professional is ready to inject the injectable material into the patient, the components of the injectable material are combined by the combining and/or delivery system 100 to be combined into (e.g., to react with each other to form) a compound material having the desired characteristics for being delivered to and/or deposited at a target site by the combining and/or delivery system 100. The multi-chamber system 200 may be fluidly coupled with the injection system 300 of the combining and/or delivery system 100 to combine the injectable material and/or to deliver the injectable material to the patient. The injection system 300 may be usable independently of the multi-chamber system 200. An optional adaptor system 400 may be included with the combining and/or delivery system 100 to adapt the injection system 300 for use with an off-the-shelf (e.g., pre-existing, separately sold, commercially available, etc.) device used to perform a procedure, treatment, pretreatment, etc., prior to or after delivery of the injectable material to the target site, as described in further detail below.

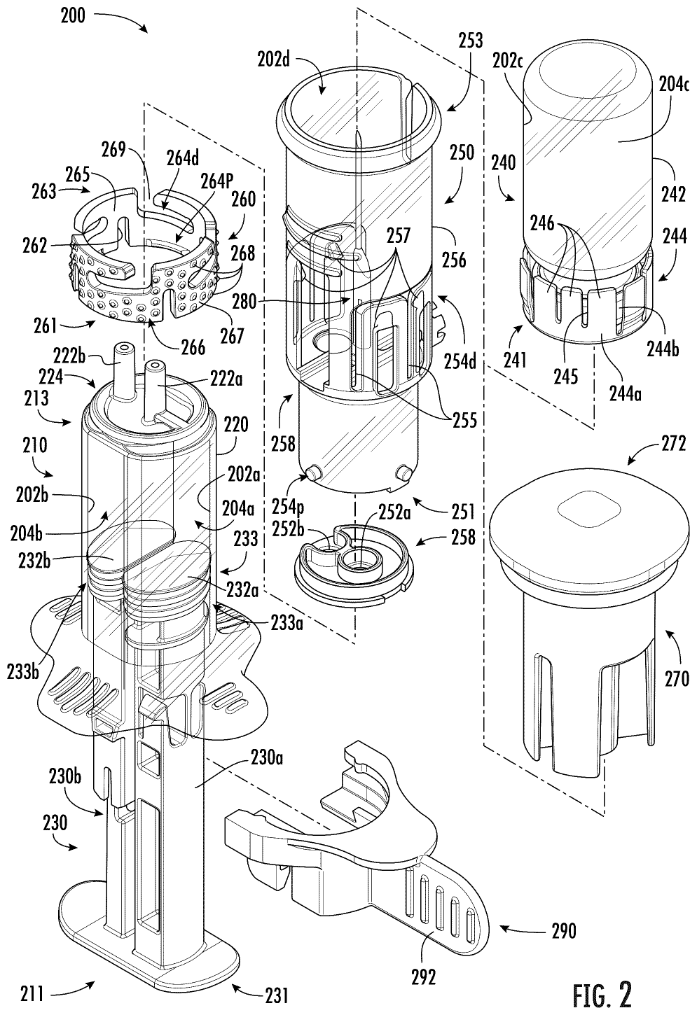

Various elements of the multi-chamber system 200 may be appreciated with reference to FIG. 2. The multi-chamber system 200 includes a device with at least one chamber for containing a component of an injectable material to be delivered to a patient. In the example of an embodiment illustrated in FIG. 2, the multi-chamber system 200 includes a multi-chamber device 210 having a barrel 220 defining a first chamber 202a and a second chamber 202b of the multi-chamber system 200. In some embodiments, the chambers 202a, 202b have substantially symmetrical cross-sectional shapes, such as half-circles if within a barrel 220 having a substantially circular cross-section, although the configurations need not be so limited. For instance, the chambers 202a, 202b may have different volumes, cross-sectional shapes, etc., such as may be dictated by the properties of the components to be contained therein. The first chamber 202a is defined to be separate and fluidly-isolated from the second chamber 202b. For instance, the contents of the first chamber 202a, e.g., a first component 204a of an injectable material, may react with the contents of the second chamber 202b, e.g., a second component 204b of an injectable material, when combined. In some embodiments, the first component 204a is combined with a third component 204c of the injectable material, such as to form a precursor 204d, and the second component 204b may react with the precursor 204d (e.g., to accelerate reaction of the first component 204a and the third component 204c). It may be desirable to keep these components separate until ready for delivery to a patient. To avoid unintentional premature combination, the contents of the chambers 202a, 202b are maintained separate from each other by the fluid isolation of the chambers 202a, 202b. When (e.g., only when) a medical professional is ready to deliver the injectable material (e.g., as a combined injectable material), the components within the chambers 202a, 202b may be combined and allowed to react with each other. The various devices, systems, and method of the present disclosure prevent unintentional combination of the first component 204a and the second component 204b, such as before the medical professional is ready to deliver the injectable material (to be formed from the first component 204a and the second component 204b) to a patient as a combined injectable material. Additionally or alternatively, the various devices, systems, and method of the present disclosure facilitate case and accuracy of combining of components to form an injectable material at the appropriate time.

The example of an embodiment of a multi-chamber device 210 illustrated in FIG. 1 further includes a plunger assembly 230 extending proximally from the barrel 220 (towards the proximal end 211 of the multi-chamber device 210). The plunger assembly 230 is movable, such as axially/longitudinally slidable (e.g., along the longitudinal axis LA), with respect to the barrel 220 to move (e.g., eject) materials out of and/or to move (e.g., aspirate) materials into chambers within the barrel 220. In the illustrated example of an embodiment, the plunger assembly 230 has a first plunger rod 230a movable/slidable with respect to the first chamber 202a and a second plunger rod 230b movable/slidable with respect to the second chamber 202b. In the illustrated example of an embodiment, distal movement of the plunger rods 230a, 230b moves materials out of the first chamber 202a and the second chamber 202b, respectively, via a first nozzle 222a and a second nozzle 222b, respectively, extending distally from the distal end 223 of the barrel 220. Conversely, proximal movement of the plunger rods 230a, 230b moves materials into the first chamber 202a and the second chamber 202b, respectively, via the first nozzle 222a and the second nozzle 222b, respectively. It will be appreciated that the plunger rods 230a, 230b may move independently of each other. Optionally, the first plunger rod 230a has a plunger stopper 232a at the distal end 233a thereof, and/or the second plunger rod 230b has a plunger stopper 232b at the distal end 233b thereof. The plunger stoppers 232a, 232b may be formed of an appropriate sealing material (e.g., an elastomeric material) known to those of ordinary skill in the art for retaining the first component 204a within the first chamber 202a and the second component 204b within the second chamber 202b.

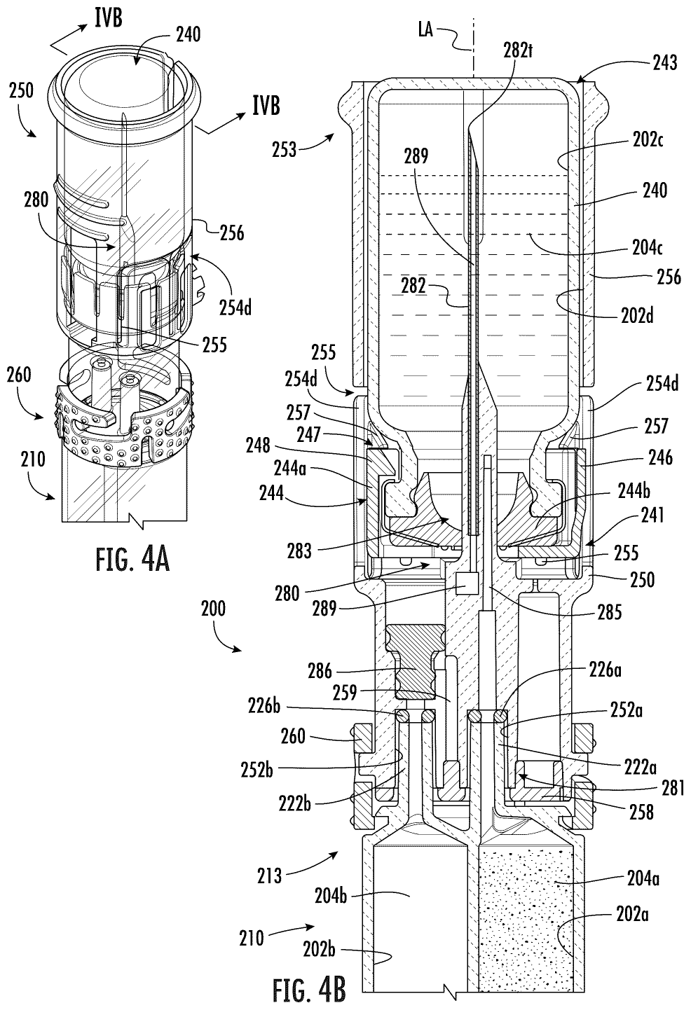

The multi-chamber system 200 optionally includes a third chamber 202c for containing an additional, third component 204c. The third component 204c may be combinable with the first component 204a contained within the first chamber 202a. The third chamber 202c optionally is defined in a separate device 240, formed and provided separately from the multi-chamber device 210, such as illustrated in FIG. 2. For the sake of convenience, and without intent to limit, the separate device is referenced herein as a vial 240, having a vial body 242 defining the third chamber 202c (which may alternately be referenced herein as a vial chamber 202c), and a vial cap 244 closing an open end 241 of the vial body 242 to contain (e.g., seal) the third component 204c within the third chamber 202c. The vial cap 244 may be a two-part cap, with an outer cap 244a holding a plug or stopper 244b with respect to the open end 241 of the vial body 242, as may be appreciated with reference to FIG. 4B, illustrating a cross-sectional view along line IVB-IVB of FIG. 4A (discussed in further detail below). The stopper 244b may be formed of a suitable material known to those of ordinary skill in the art as capable of sealing the third component 204c within the third chamber 202c defined within the vial 240 and optionally also allowing access therethrough, as described in further detail below. For example, the stopper 244b may be formed of an elastic material capable of being pierced to allow fluid communication therethrough, such as described in further detail below.

A vial adaptor 250 is configured to facilitate holding/coupling of the vial 240 with respect to the multi-chamber device 210 to transfer materials therebetween, in a manner described in further detail below. The example of an embodiment of a vial adaptor 250 illustrated in FIG. 2 defines a fourth chamber 202d of the multi-chamber system 200 opening to the distal end 253 of the vial adaptor 250. The fourth chamber 202d may be configured as a vial-receiving chamber configured to receive the vial 240 and to hold the vial 240 in alignment with the multi-chamber device 210 so that the multi-chamber device 210 and the vial 240 may be fluidly coupled for transferring and/or exchanging material therebetween. For instance, the vial adaptor 250 may facilitate alignment and/or fluid sealing between the vial adaptor 250 and the multi-chamber device 210 so that materials may be transferred between a vial 240 (within the vial adaptor 250) and the multi-chamber device 210 without leakage, and/or without significant manipulation of components of the combining and/or delivery system 100. More particularly, the vial adaptor 250 facilitates fluid coupling of a first chamber 202a of a multi-chamber device 210 formed in accordance with various principles of the present disclosure with a separate chamber 202c, and the combination of the components 204a, 204c contained within the chambers 202a, 202c. The vial adaptor 250 may include a first nozzle port 252a positioned and configured to be fluidly coupled with (e.g., to receive and to be in fluid communication with) the first nozzle 222a of the barrel 220 of the multi-chamber device 210, and a second nozzle port 252b positioned and configured to be fluidly coupled with (e.g., to receive and to be in fluid communication with) the second nozzle 222b of the barrel 220 of the multi-chamber device 210, as may be appreciated with reference to FIG. 4B and as discussed in further detail below.

The example of an embodiment of a multi-chamber system 200 illustrated in FIG. 2 further includes a sleeve lock 260 configured to facilitate secure coupling of the vial adaptor 250 with the multi-chamber device 210. For instance, the example of an embodiment of a sleeve lock 260 illustrated in FIG. 2 is configured to stabilize the multi-chamber device 210 and the vial adaptor 250 with respect to each other, such as to maintain fluid coupling therebetween without leakage. For instance, the sleeve lock 260 may be generally cylindrical, such as in the shape of a ring or collar, and may alternately be referenced as a locking ring, locking collar, adaptor ring, adaptor collar, etc., without intent to limit. The length of the sleeve lock 260 (e.g., along the longitudinal axis LA) may be selected to hold the vial adaptor 250 against lateral movement (transverse to the longitudinal axis LA) with respect to the multi-chamber device 210 when coupled thereto. Optionally, the sleeve lock 260 is configured to be mounted with respect to (e.g., and carried on) the multi-chamber device 210, such as described in further detail below. In such embodiment, the sleeve lock 260 includes flex slots 262 facilitating mounting of the sleeve lock 260 on at least one of the multi-chamber device 210 or the vial 240, such as by an interference fit.

As may be appreciated with reference to the example of an embodiment illustrated in FIG. 2, the sleeve lock 260 optionally includes one or more (e.g., 1-4, or more) engaging elements 264 configured to engage at least one of the multi-chamber device 210 or the vial adaptor 250 to facilitate coupling of the multi-chamber device 210 and the vial adaptor 250. For instance, the example of an embodiment of a sleeve lock 260 illustrated in FIG. 2 includes one or more (e.g., 1-4, or more) proximal engaging elements 264p configured to engage the multi-chamber device 210, and one or more (e.g., 1-4, or more) distal engaging elements 264d configured to engage the vial adaptor 250. More particularly, in the illustrated example of an embodiment, the one or more proximal engaging elements 264p are configured to engage with one or more corresponding sleeve-lock-engaging elements 224 on the multi-chamber device 210, such as on the barrel 220 of the multi-chamber device 210. Similarly, the one or more (e.g., 1-4, or more) distal engaging elements 264d are configured to engage with one or more (e.g., 1-4, or more) proximal sleeve-lock-engaging elements 254p on the vial adaptor 250. The sleeve lock 260 may thereby be coupled to the distal end 213 of the multi-chamber device 210 and the proximal end 251 of the vial adaptor 250 to couple the vial adaptor 250 to the distal end 213 of the multi-chamber device 210.

In the example of an embodiment of a sleeve lock 260 illustrated in FIG. 2, the one or more proximal engaging elements 264p of the sleeve lock 260 are in the form of one or more circumferentially-extending features (e.g., undercut locking tab features, ribs, shoulders, etc.) extending partially or fully around the interior surface 265 of the sleeve lock 260 and engaging with corresponding sleeve-lock-engaging elements 224 in the form of corresponding features (e.g., a groove, rib, circumferentially-extending shoulder, etc.) extending partially or fully circumferentially around the barrel 220 of the multi-chamber device 210 adjacent the distal end 213 of the multi-chamber device 210. As such, the sleeve lock 260 may be rotatably mounted on the distal end 213 of the multi-chamber device 210. The sleeve lock 260 may be snap-fitted with the barrel 220 of the multi-chamber device 210 to be coupled thereto and mounted/carried on the distal end 213 thereof. The flex slots 262 may facilitate mounting of the sleeve lock 260 on the distal end 253 of the barrel 220, such as by radially expanding the sleeve lock 260 and the proximal engaging elements 264p to be advanced axially over the sleeve-lock-engaging elements 224 on the barrel 220.

In contrast, the distal engaging elements 264d of the example of an embodiment of a sleeve lock 260 illustrated in FIG. 2 facilitate removable coupling of the vial adaptor 250 with the sleeve lock 260 and thus with the multi-chamber device 210. More particularly, in the example of an embodiment of a sleeve lock 260 illustrated in FIG. 2, the one or more distal engaging elements 264d of the sleeve lock 260 and the one or more proximal sleeve-lock-engaging elements 254p on the vial adaptor 250 are engaged and/or disengaged by axial and/or rotational movements (in contrast with interference fits which generally require overcoming of a threshold force). The sleeve-lock-engaging elements 254p on the vial adaptor 250 may be configured to readily be initially engaged with respect to, and/or to remain engaged with, and/or to be disengaged from the distal engaging elements 264d on the sleeve lock 260. For instance, the distal engaging elements 264d of the sleeve lock 260 may be in the form of slots, and the sleeve-lock-engaging elements 254p on the vial adaptor 250 may be in the form of bosses, projections, pins, nubs, etc. (referenced herein as projections for the sake of convenience and without intent to limit) configured to engage with the proximal engaging elements 264p. The slots forming the proximal engaging elements 264p of the sleeve lock 260 may extend from the interior surface 265 of the sleeve lock 260 only partially through the wall of the sleeve lock 260, or completely through the wall 266 of the sleeve lock 260 to the exterior surface 267 of the wall 266 of the sleeve lock 260 (such as illustrated in FIG. 2). Such configuration allows a relatively simple (e.g., a bayonet-type) connection between the sleeve lock 260 and the vial adaptor 250 facilitating ready engagement and/or disengagement of the vial adaptor 250 (and thus a vial 240 inserted or to be inserted therein) with respect to the multi-chamber device 210 (which is already coupled with the sleeve lock 260). For instance, the vial adaptor 250 and the sleeve lock 260 (optionally already mounted on the barrel 220, such as described above) may be brought together generally axially to engage the sleeve-lock-engaging elements 254p of the vial adaptor 250 with the distal engaging elements 264d of the sleeve lock 260, such as by entering the entry openings 269 of the slot-like distal engaging elements 264d illustrated in FIG. 2. The sleeve lock 260 and vial adaptor 250 may be rotated with respect to each other to cause the sleeve-lock-engaging elements 254p of the vial adaptor 250 to extend further into the distal engaging elements 264d of the sleeve lock 260 to interlock the sleeve-lock-engaging elements 254p and the distal engaging elements 264d to hold the vial adaptor 250 and sleeve lock 260 axially in place with respect to each other. Such configuration allows the multi-chamber device 210 (e.g., with the assistance of the sleeve lock 260) to be readily engaged/coupled with the vial adaptor 250 in a manner inhibiting axial separation of these components once coupled, and allowing ready disengaging/decoupling.

It will be appreciated that reverse arrangements between the sleeve lock engagement elements and the corresponding engagement elements on the multi-chamber device 210 and/or the vial adaptor 250 are within the scope of the present disclosure. For instance, ribs may be provided on the multi-chamber device 210 for engaging within grooves defined in the interior surface 265 of the sleeve lock 260, and/or projections may be provided on the vial adaptor 250 for engagement within locking slots in the vial adaptor 250. Moreover, instead of being carried on the multi-chamber device 210, the sleeve lock 260 may be carried on the vial adaptor 250, in which case the rib-groove engagement may be provided between the sleeve lock 260 and the vial adaptor 250, and the projection-slot engagement may be provided between the sleeve lock 260 and the multi-chamber device 210. In some embodiments, a rib-groove engagement is used between the sleeve lock 260 and both the multi-chamber device 210 and the vial adaptor 250. In some embodiments, a slot-projection engagement is used between the sleeve lock 260 and both the multi-chamber device 210 and the vial adaptor 250.

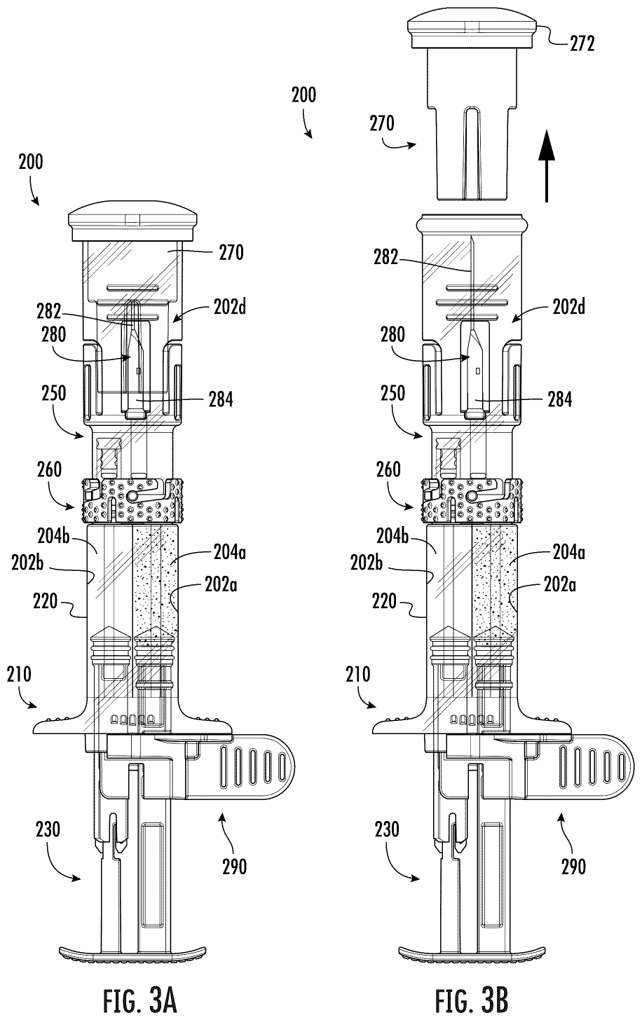

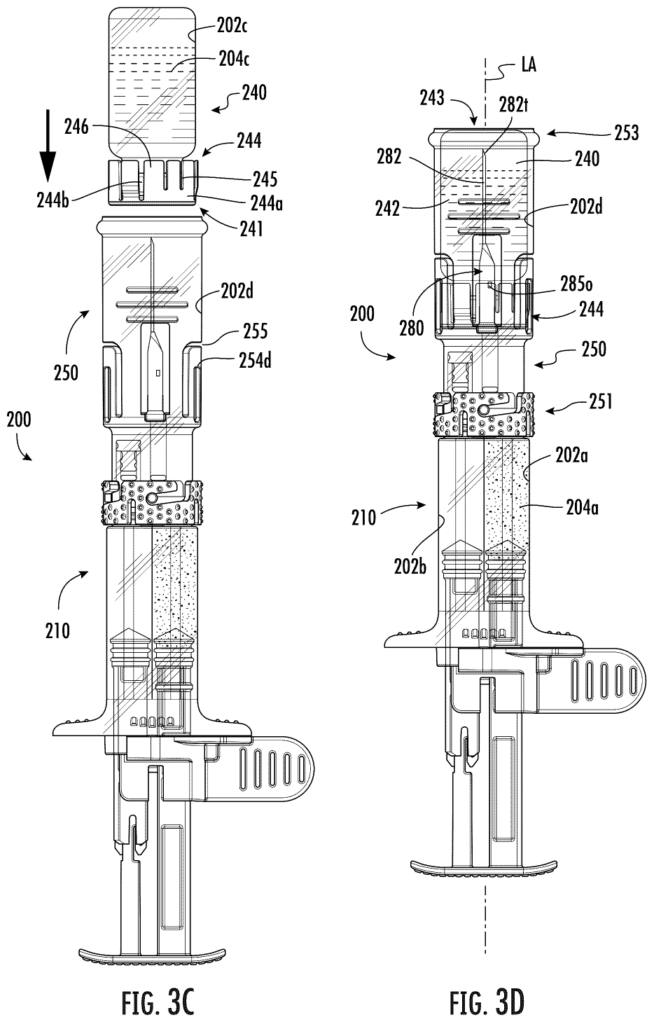

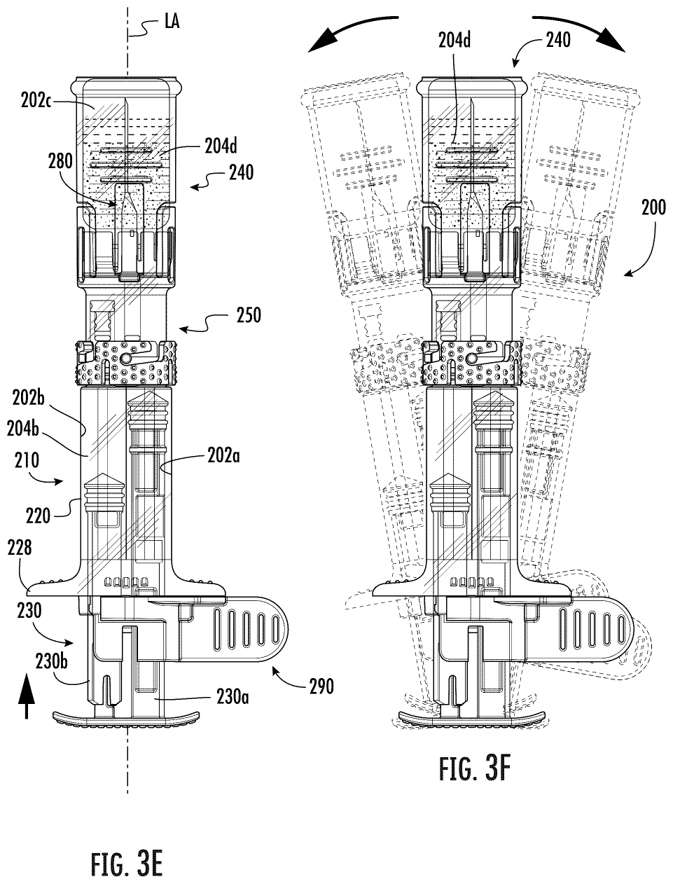

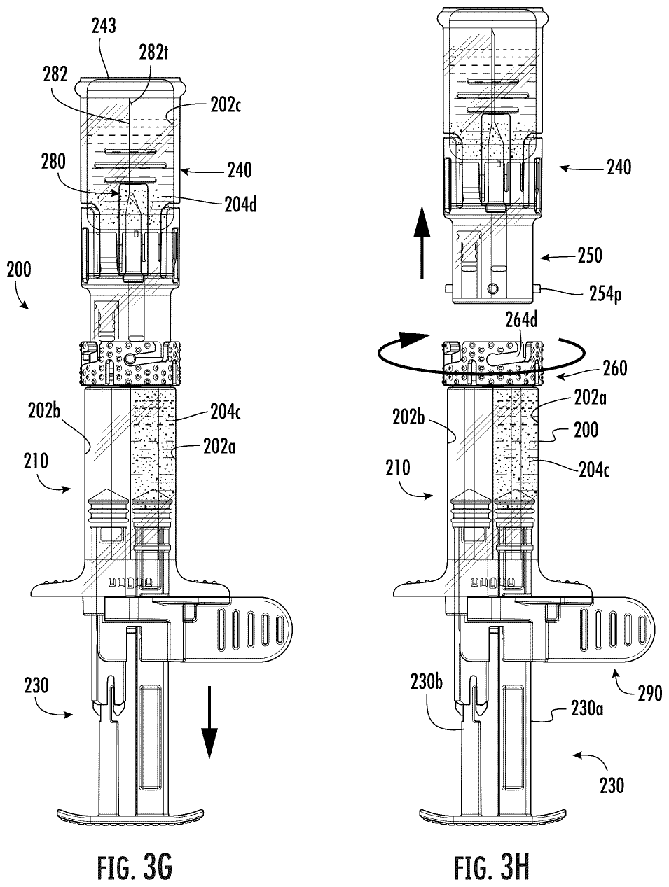

The vial adaptor 250 may be coupled with the multi-chamber device 210 with a protective cap 270 positioned within the fourth chamber 202d, prior to insertion of the vial 240 into the fourth chamber 202d of the vial adaptor 250. The protective cap 270 may be configured to protect or shield components of a fluid exchange device 280 extending within the fourth chamber 202d of the vial adaptor 250 (such as a piercing element 282 and fluid exchange spike 284, as illustrated in FIG. 3A, FIG. 3B, FIG. 4A, and FIG. 4B). The fluid exchange device 280 is configured to fluidly communicate the first chamber 202a within the barrel 220 of the multi-chamber device 210 with the third chamber 202c within the vial 240, as may be appreciated with reference to FIG. 4B (showing a cross-sectional view along line IVB-IVB of FIG. 4A), and as described in further detail below. The protective cap 270 may also protect the vial adaptor 250 from entry of unwanted matter or debris from entering the fourth chamber 202d.

The various interactions of the various components of a multi-chamber system 200 formed in accordance with various principles of the present disclosure and illustrated in FIG. 2 may be appreciated with reference to FIG. 3A-FIG. 3H. The multi-chamber device 210, vial adaptor 250, and sleeve lock 260 are illustrated in FIG. 3A assembled together for delivery of the first component 204a and the second component 204b (within respective chambers 202a, 202b defined within the barrel 220 of the multi-chamber device 210) to a patient. The optional protective cap 270 is illustrated in FIG. 3A positioned within the fourth chamber 202d of the vial adaptor 250. Additionally, an optional retainer 290 is illustrated as coupled with the plunger assembly 230 (e.g., mounted thereon) to limit movement of the plunger assembly 230 with respect to the barrel 220 to prevent inadvertent ejection of materials from the multi-chamber device 210.

To prepare the multi-chamber system 200 for combining the first component 204a with a third component 204c (such as delivered within the optional vial 240), the protective cap 270 is separated from the vial adaptor 250 (e.g., removed from the fourth chamber 202d), as illustrated in FIG. 3B. Optionally, the protective cap 270 includes a grasping section 272, such as a radially-outwardly projecting flange, configured to facilitate grasping and pulling on the protective cap 270 to remove the protective cap 270 from the vial-receiving fourth chamber 202d defined in the vial adaptor 250. The vial 240 is then aligned with the vial adaptor 250 for insertion into the fourth chamber 202d, as illustrated in FIG. 3C.

In FIG. 3D, the vial 240 is illustrated positioned within the fourth chamber 202d of the vial adaptor 250. The vial adaptor wall 256 facilitates alignment of the vial 240 with respect to the multi-chamber device 210 and the vial adaptor 250, as may be appreciated with reference to the cross-sectional view illustrated in FIG. 4B (along line IVA-IVA of FIG. 4A). The respective longitudinal axes of the vial 240, the multi-chamber device 210, and the vial adaptor 250 may be generally aligned with one another as well as with the longitudinal axis LA of the multi-chamber system 200. The vial 240 and/or vial adaptor 250 may include further features facilitating alignment of the vial 240 with the multi-chamber device 210 and the vial adaptor 250. In some embodiments, the outer diameter of the outer cap 244a is greater than the outer diameter of the vial body 242 to facilitate alignment of the vial 240 within the fourth chamber 202d of the vial adaptor 250. Additionally or alternatively, the outer cap 244a of the vial cap 244 may include one or more (e.g., 1-4, or more) flexible flanges 246 (such as defined by slits 245, such as illustrated in FIG. 2 and FIG. 3C) to further facilitate alignment of the vial 240 within the vial adaptor 250 and with respect to other components of the multi-chamber system 200. Additionally or alternatively, the vial adaptor 250 may include one or more (e.g., 1-4, or more) vial-engaging elements 254d (such as defined by slits 255 in the vial adaptor wall 256, such as illustrated in FIG. 2 and FIG. 3C and FIG. 4B and FIG. 4B) configured to engage the vial 240, such as via the vial cap 244. For instance, one or more (one to all) of the vial-engaging elements 254d may have radially-inwardly extending shoulders 257 configured to engage a respective free end 247 of a flexible flange 246 of the outer cap 244a of the vial cap 244, such as may be seen with reference to FIG. 4B.

Alignment of the vial 240 within the vial adaptor 250 facilitates alignment of the vial 240 with the fluid exchange device 280 between the vial 240 and the multi-chamber device 210. The fluid exchange device 280 of the combining and/or delivery system 100 may be considered to be part of an overall fluid exchange system which includes components of the multi-chamber device 210, the vial adaptor 250, and even the vial 240 which are configured and engaged to facilitate combining of the first component 204a, contained within the first chamber 202a defined in the barrel 220 of the multi-chamber device 210, with the third component 204c, contained within the third chamber 202c defined in the vial 240. In the example of an embodiment illustrated in FIG. 4B, the fluid exchange device 280 includes a piercing element 282, such as a needle, extending from a fluid exchange spike 284 configured to fluidly communicate the first chamber 202a and the third chamber 202c. For instance, a proximal end of the piercing element 282 (along the proximal end 281 of the fluid exchange device 280) may be mounted with respect to (e.g., on or in) the fluid exchange spike 284 and may extend distally toward the distal end 283 of the fluid exchange device 280 and end in a sharpened distal tip 282t. The piercing element 282 and its sharpened distal tip 282t may be positioned and configured to pierce the stopper 244b of the vial cap 244. It will be appreciated that terms such as pierce, puncture, etc., (including other grammatical forms thereof) may be used interchangeably herein without intent to limit unless otherwise specified. In the example of an embodiment illustrated in FIG. 4B, the outer cap 244a extends around the circumference/periphery of the stopper 244b leaving a generally central region of the stopper 244b exposed for access therethrough, such as by the piercing element 282, into the third chamber 202c within the vial 240. In some embodiments, the stopper 244b has a reduced wall thickness (e.g., along a generally central region of the stopper 244b, such as illustrated in FIG. 4B) for facilitating piercing thereof by the piercing element 282. It will be appreciated that the piercing element 282, and its sharpened distal tip 282t, may be positioned to be generally aligned with a thinner wall portion of the stopper 244b to facilitate piercing of the thinner wall portion of the stopper 244b by the piercing element 282, and the various alignment features described above may facilitate/foster such alignment of the piercing element 282 with a thinner wall portion of the stopper 244b.

Once the stopper 244b has been pierced or punctured, the wider fluid exchange spike 284 (generally having a larger cross-sectional area than that of the piercing element 282), from which the piercing element 282 extends, may more readily extend through the stopper 244b of the vial 240. The first chamber 202a and the third chamber 202c of the multi-chamber system 200 are fluidly communicated via a fluid exchange lumen 285 extending through the fluid exchange spike 284. Optionally the fluid exchange lumen 287 communicates with the third chamber 202c via a transverse outlet 2850, such as indicated in FIG. 3D. In the example of an embodiment illustrated in FIG. 4B, the fluid exchange lumen 285 of the fluid exchange spike 284 is in fluid communication with the first chamber 202a via the first nozzle 222a of the barrel 220 of the multi-chamber device 210. More particularly, the first nozzle 222a may be seated with respect to the vial adaptor 250 via (e.g., by extending into) a first nozzle port 252a, and the first nozzle port 252a may be configured to facilitate fluid communication of the first nozzle 222a and the fluid exchange lumen 285 of the fluid exchange spike 284. Optionally, as illustrated in FIG. 2 and FIG. 4B, the first nozzle port 252a and the second nozzle port 252b are defined in/by a vial adaptor base 258. The vial adaptor base 258 may be formed integrally with (e.g., as a single piece with) or separately from the vial adaptor wall 256. The fluid exchange spike 284 may be formed integrally with (e.g., as a single piece with) or separately from the vial adaptor base 258. Seals 226a, 226b (e.g., O-rings) may be provided, respectively, between the nozzles 222a, 222b and the nozzle ports 252a, 252b, such as to seal against leakage, such as in a manner such as known to those of ordinary skill in the art. Optionally, a valve 286 is positioned in or adjacent the second nozzle port 252b in the vial adaptor 250 to prevent unwanted flow of materials between the third chamber 202c (within the vial 240) and the second chamber 202b (within the barrel 220 of the multi-chamber device 210), to ensure the correct components within the multi-chamber device 210 are mixed with the component within the vial 240 via the fluid exchange device 280, and to minimize if not eliminate mixing errors.

Once the vial 240 is seated within the fourth chamber 202d of the multi-chamber system 200 defined by the vial adaptor 250, and the third chamber 202c defined within the vial 240 is in fluid communication with the first chamber 202a defined within the barrel 220 of the multi-chamber device 210, such as via the fluid exchange device 280, the plunger assembly 230 may be advanced distally with respect to the barrel 220, as illustrated in FIG. 3E. A finger grip 228 (e.g., one or more radially-outwardly extending flanges) may be provided on the barrel 220 to facilitate grasping and/or holding of the barrel 220 to move the plunger assembly 230 relative to the barrel 220. As may be appreciated with reference to FIG. 3E, the plunger assembly 230 may be configured such that the first plunger rod 230a and the second plunger rod 230b are independently advanceable. More specifically, the first plunger rod 230a and the second plunger rod 230b do not necessarily advance together. Such configuration of the plunger assembly 230 allows the first plunger rod 230a to eject the first component 204a out of the first chamber 202a and into the vial 240 without the second plunger rod 230b ejecting the second component 204b out of the second chamber 202b. For instance, the first plunger rod 230a may extend the full, or close to the full, longitudinal extent of the first chamber 202a (along a longitudinal axis LA, such as a longitudinal axis of the barrel 220, or substantially parallel to longitudinal axis of the barrel 220) to expel all or most of the first component 204a out from the first chamber 202a and to inject the first component 204a into the third chamber 202c for combination with the third component 204c therein.

In some embodiments, the second plunger rod 230b may be partially advanced distally into the second chamber 202b when the first plunger rod 230a is substantially fully advanced to expel the first component 204a out from the first chamber 202a. In other words, the second plunger rod 230b may be advanced along only a portion of the longitudinal extent of the second chamber 202b (along a longitudinal axis LA, such as a longitudinal axis of the barrel 220, or substantially parallel to a longitudinal axis of the barrel 220), typically shorter than the longitudinal extent along which the first plunger rod 230a advances. Partial advancement of the second plunger rod 230b may be beneficial and/or desirable to expel any surplus of the second component 204b, and/or any air bubbles, out from the second chamber 202b into a purge channel 259 (illustrated in FIG. 4B) within the vial adaptor 250. The purge channel 259 may be in fluid communication with and/or expand into a larger catch reservoir defined within the vial adaptor 250, such as by the vial adaptor wall 256 and the vial adaptor base 258. The purge channel 259 may expel excess material out from the second chamber 202b and into the purge channel 259 (and optional larger catch reservoir) so that the material is collected within the combining and/or delivery system 100 and not expelled outside the combining and/or delivery system 100 (e.g., onto the medical professional, the patient, the floor, etc.). The valve 286 in the fluid path between the second nozzle 222b and the second nozzle port 252b (see, e.g., FIG. 4B) may regulate flow of the second component 204b from the second chamber 202b into the purge channel 259. The valve 286 may be a one-way valve allowing purging of excess material out of the second chamber 202b, but preventing the third component 204c (from the third chamber 202c) from entering the second chamber 202b.

Once the first component 204a has been injected into the third chamber 202c, the multi-chamber system 200 may be shaken, such as schematically illustrated in FIG. 3F, such as to facilitate mixing of the first component 204a and the third component 204c within the vial 240. The combined first component 204a and third component 204c are then aspirated back into the first chamber 202a by retracting the plunger assembly 230 proximally, as illustrated in FIG. 3G. For the sake of convenience, and without intent to limit, the combination of the first component 204a and the third component 204c is referenced herein as a precursor 204d. It will be appreciated that in some instances, the entire content of the vial 240 (the combined first component 202a and third component 202c) is not aspirated out of the vial. 240. In some embodiments, the second component 204b (within the second chamber 202b) may react with the precursor 204d if combined therewith (e.g., the second component 204b may be an accelerant for completing a reaction between the first component 204a and the second component 204b). As such, as described above with respect to first component 204a and the second component 204b, the precursor 204d and the second component 204b may be maintained separate, such as by fluid isolation of the first chamber 202a and the second chamber 202b, until the user is ready for intentional combination of such components (e.g., when ready for delivery to a patient).

As the precursor 204d (formed upon combining the first component 204a and the second component 204b within the vial 240) is withdrawn from the vial 240, it may be desirable to avoid creation of a vacuum within the vial 240. In some embodiments, the fluid exchange device 280 is configured to provide venting, and the vial adaptor 250 in such embodiments may thus be considered a vented vial adaptor 250. For instance, the piercing element 282 of the fluid exchange device 280 may have a venting lumen 287 defined therethrough opening at the sharpened distal tip 282t and extending proximally through the fluid exchange device 280 into fluid communication with a venting passage 289 defined in the fluid exchange spike 284 (see, e.g., FIG. 4B) in fluid communication with atmospheric air. The piercing element 282 may be sufficiently long enough to extend within the third chamber 202c distally towards the closed end 243 of the vial 240, when the vial 240 is inverted within the vial adaptor 250, so that the sharpened distal tip 282t of the piercing element 282 passes beyond the contents of the vial 240 (e.g., the precursor 204d) and into the space between the contents of the vial 240 and the closed end 243 of the vial 240. As the contents of the vial 240 are withdrawn from the vial 240, atmospheric air may pass through the venting lumen 287 and into the space within the third chamber 202c between the contents of the vial 240 and the closed end 243 of the vial 240.

Once the combined first component 204a and third component 204c have been aspirated into the first chamber 202a, the vial 240 and the vial adaptor 250 are withdrawn from the multi-chamber device 210, as illustrated in FIG. 3H. As noted above, the sleeve lock 260 may be configured to be rotatably mounted on the distal end 213 of the multi-chamber device 210. In such example of an embodiment, rotation of the sleeve lock 260 and the vial adaptor 250 with respect to each other (and optionally with respect to the multi-chamber device 210 as well) allows the sleeve-lock-engaging elements 254p of the vial adaptor 250 to rotate and then move generally axially out of the distal engaging elements 264d of the sleeve lock 260 to release the vial adaptor 250 from the sleeve lock 260 for separation from the multi-chamber device 210. It will be appreciated that such movement may be generally opposite a movement of the vial adaptor 250 and its sleeve-lock-engaging elements 254p relative to the sleeve lock 260 to couple the vial adaptor 250 with the multi-chamber device 210. Optionally, seals may be provided to seal the connection between the nozzles 222a, 222b of the multi-chamber device 210 and the corresponding nozzle ports 252a, 252b of the vial adaptor 250 and/or to provide a biasing force to hold the multi-chamber device 210 and the vial adaptor 250 in engagement to resist inadvertent rotation therebetween, such as in a manner known to those of ordinary skill in the art. Moreover, it will be appreciated that the above-described configuration of engagement elements provides a relatively simple engagement and alignment of the multi-chamber device 210 and the vial adaptor 250 for combination of the first component 204a and the third component 204c within the vial 240. Finally, it will be appreciated that in some instances, the entire content of the vial 240 (the combined first component 204a and third component 204c) is not aspirated out of the vial 240. With the first chamber 202a now containing the precursor 204d, and the second chamber 202b having optionally been purged of excess second component 204b, the multi-chamber device 210 is ready to deliver the injectable materials contained within the first chamber 202a and the second chamber 202b to the patient.

Although the example of an embodiment of a multi-chamber system 200 illustrated in FIG. 2 has a multi-chamber device 210 configured to deliver a first component separate from a second component for combining upon delivery to a patient, it will be appreciated that various principles of the present disclosure may be applied to a single-chamber device. The single-chamber device may be any device, such as known to those of ordinary skill in the art, similar to the multi-chamber device 210, but with only one chamber defined within the barrel 220 (illustration thereof not being considered necessary for full understanding thereof by those of ordinary skill in the art). Components within the single-chamber device may be combined with components within a separately-provided chamber (e.g., an additional component provided in a distinct and/or separate device with a chamber for such additional component, such as a vial separate from the single-chamber device). Alternatively, the multi-chamber device 210 may deliver a first component 204a and a second component 204b for delivery to a patient without combining either component 204a, 204b with a separate component 204c delivered separately from the multi-chamber device 210 (e.g., in a vial 240). Furthermore, it will be appreciated that reference is made to combining the first component 204a with the third component 204c, but the separate component 204c may instead be combined with the second component 204b. It will be appreciated that various principles of the present disclosure may be applied to other combinations and variations of devices, chambers, components, etc.

As noted above, a combining and/or delivery system 100 formed in accordance with various principles of the present disclosure includes a device containing material to be delivered to a patient (in the example of an embodiment illustrated in FIG. 1, a multi-chamber device 210) and a separate device configured to deliver the material to the patient (in the example of an embodiment illustrated in FIG. 1, an injection system 300). For instance, the device containing the material may not be configured to deliver the material directly to a patient, but, instead, is fluidly couplable with an injection system which is configured to deliver the material directly to a patient. In accordance with various principles of the present disclosure, a combining and/or delivery system 100 is configured to facilitate coupling of the multi-chamber device 210 with the injection system 300, such as with the use of a sleeve lock 260 such as described above. Thus, the sleeve lock 260 of the multi-chamber system 200 may be used not only to facilitate coupling of the multi-chamber device 210 with an optional vial adaptor 250 as described above (if a separate third chamber 202c is to be fluidly coupled with the chambers 202a, 202b of a single-chamber device or a multi-chamber device 210), but also to facilitate coupling of the multi-chamber device 210 (or a single-chamber device) with the injection system 300.

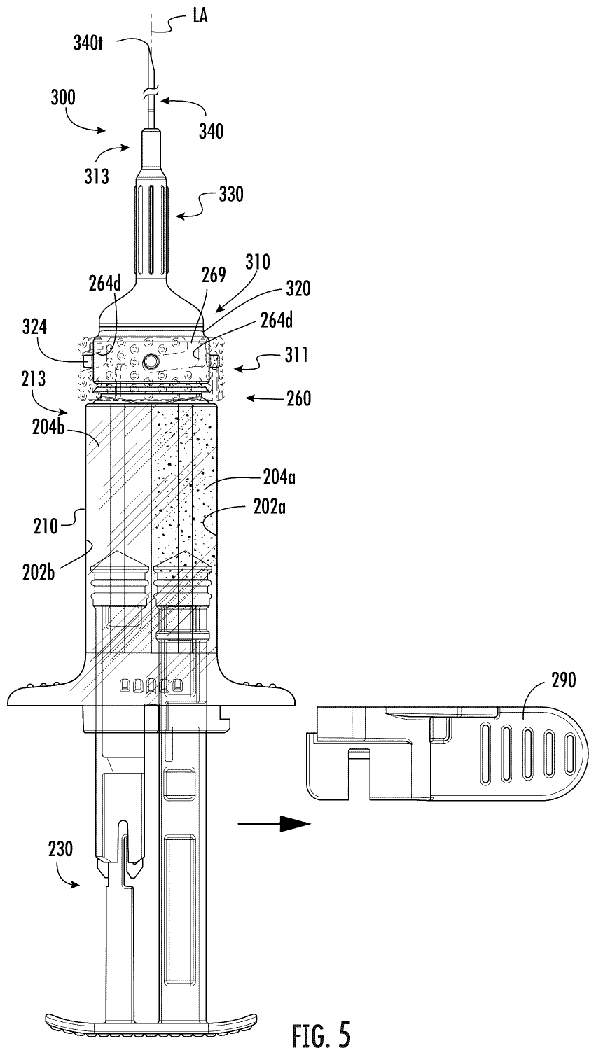

As described above, and as may be appreciated with reference to FIG. 3H, the sleeve lock 260 may be mounted on the multi-chamber device 210 and carried therewith to the injection system 300 (e.g., after optional coupling with a vial adaptor 250 and subsequent separation therefrom, as described above). The optional retainer 290 may also be mounted on the multi-chamber device 210 to avoid inadvertent ejection of materials within the chambers 204a, 204b within the barrel 220 until the multi-chamber device 210 is coupled with the injection system 300. The multi-chamber device 210 is fluidly coupled with the injection system 300 with the assistance of the sleeve lock 260, and the optional retainer 290 is withdrawn, such as illustrated in FIG. 5, to ready these components of the combining and/or delivery system 100 for delivery of an injectable material to a patient.

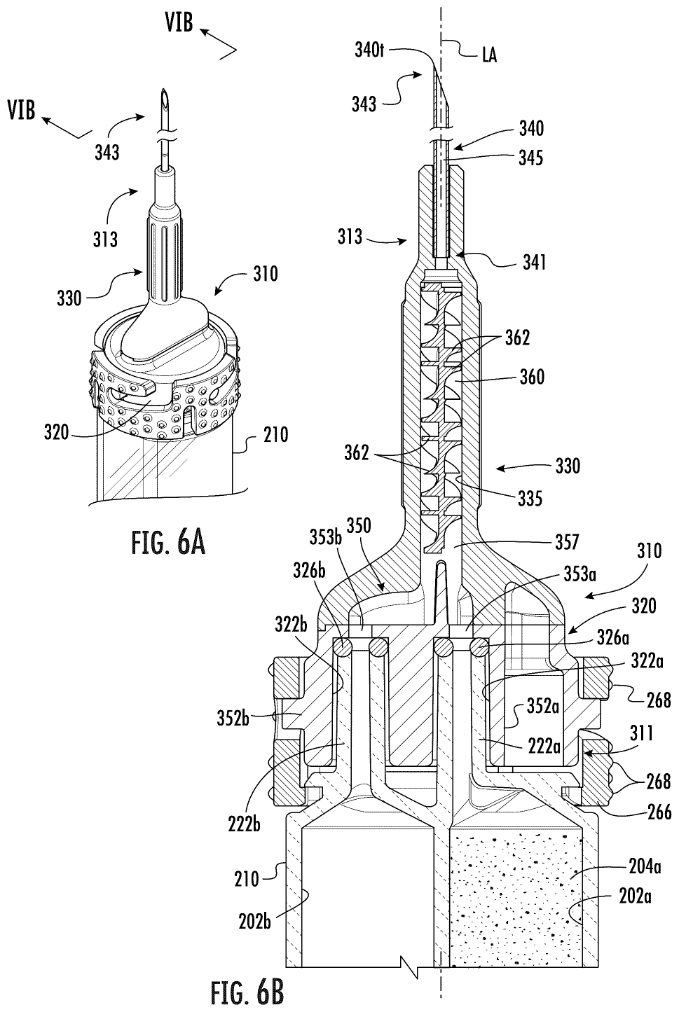

Similar to the vial adaptor 250, the injection system 300 may be configured to facilitate fluid coupling with the multi-chamber device 210 with the assistance of the sleeve lock 260. For instance, in the example of an embodiment illustrated in FIG. 5 and FIG. 6B, the injection system 300 includes a base 310 with a proximal end 311 defining a connector portion 320 configured to be fluidly coupled with the multi-chamber device 210, such as with the assistance of the sleeve lock 260. As may be appreciated with reference to the cross-sectional view along line VIB-VIB of FIG. 6A, as illustrated in FIG. 6B, the injection system connector portion 320 defines nozzle ports 322a, 322b configured to facilitate fluid coupling of the multi-chamber device 210 with the injection system 300 by respectively mating with the nozzles 222a, 222b of the barrel 220 of the multi-chamber device 210. For instance, as illustrated in the cross-sectional view illustrated in FIG. 6B, the first injection system nozzle port 322a may be configured to receive the first multi-chamber device nozzle 222a, with the first nozzle 222a extending into and/or seating with respect to the first nozzle port 322a. Similarly, the second injection system nozzle port 322b may be configured to receive the second multi-chamber device nozzle 222b, with the second nozzle 222b extending into and/or seating with respect to the second nozzle port 322b. Seals 326a, 326b (e.g., O-rings) may be provided, respectively, between the nozzles 222a, 222b and the nozzle ports 322a, 322b, such as to seal against leakage. The sleeve lock 260 may not only facilitate alignment of the multi-chamber device 210 and the injection system 300, such as to facilitate alignment of the nozzles 222a, 222b with the nozzle ports 322a, 322b, but also to couple the multi-chamber device 210 with the injection system 300 to facilitate cooperative engagement and use thereof, such as during transfer of the contents of the multi-chamber device 210 to the injection system 300 for delivery to a patient.

To facilitate coupling of the injection system 300 with the multi-chamber device 210 with the assistance of the sleeve lock 260, the injection system connector portion 320 includes one or more (e.g., 1-4, or more) sleeve-lock-engaging elements 324 configured to engage with the one or more distal engaging elements 264d of the sleeve lock 260 carried by the multi-chamber device 210. In the example of an embodiment illustrated in FIG. 5, the multi-chamber device 210 may be coupled with the injection system 300 with the assistance of the sleeve lock 260 by axial movement of the sleeve lock 260 with respect to the injection system 300 to engage the sleeve-lock-engaging elements 324 of the injection system 300 with the distal engaging elements 264d of the sleeve lock 260. For instance, the sleeve-lock-engaging elements 324 may generally axially (e.g., along the longitudinal axis LA) enter the entry openings 269 of the slot-like distal engaging elements 264d. The sleeve lock 260 and the injection system 300 may then be rotated with respect to each other (and optionally with respect to the multi-chamber device 210 as well) to complete engagement of the sleeve-lock-engaging elements 324 with the distal engaging elements 264d. Such configuration provides a relatively simple engagement and alignment of the multi-chamber device 210 and the injection system 300 for delivery of the contents of the multi-chamber device 210, via the injection system 300, to a patient. Optionally, the seals may be provided to seal the connection between the multi-chamber device 210 and the injection system 300 and/or to provide a biasing force to hold the multi-chamber device 210 and the injection system 300 in engagement to resist inadvertent rotation therebetween, such as in a manner known to those of ordinary skill in the art.

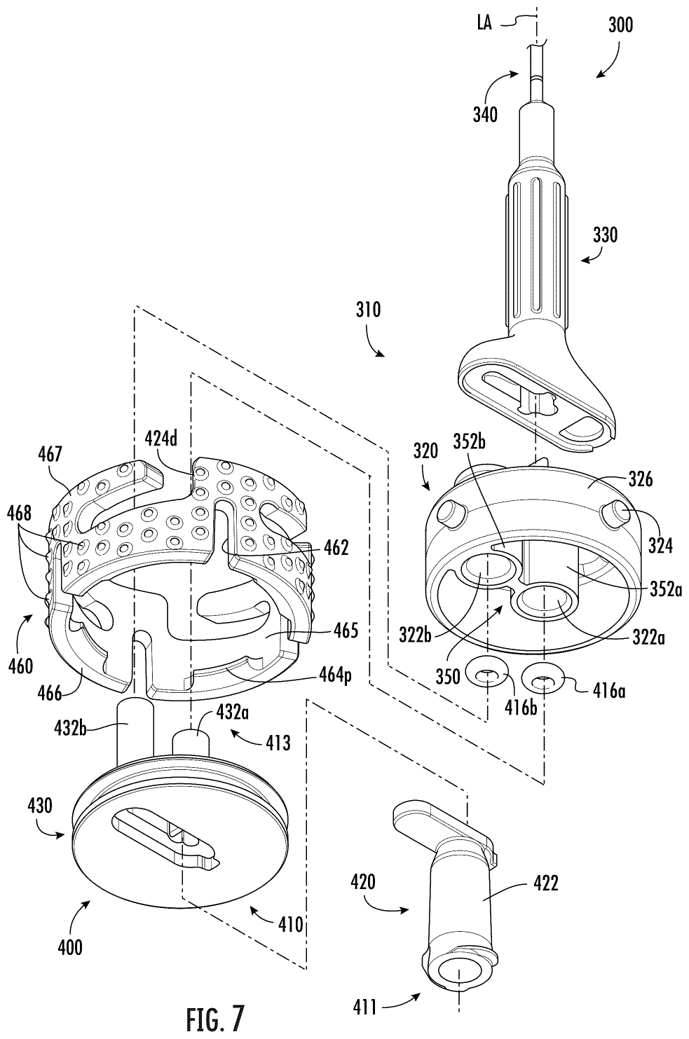

Once the multi-chamber device 210 is securely coupled with the injection system 300, such as with the assistance of the sleeve lock 260, as illustrated in FIG. 5, the chambers 202a, 202b of the multi-chamber device 210 are fluidly coupled with the injection system 300 to combine the second component 204b and the precursor 204d (or a first component 204a if not mixed with a third component 204c to form a precursor 204d) for delivery to a patient. More particularly, in the example of an embodiment illustrated in FIG. 5, the distal end 313 of the injection system base 310 defines a support portion 330 configured to support a proximal end 341 of a material delivery device 340, as may be seen in the cross-sectional view of FIG. 6B. It will be appreciated that the injection system connector portion 320 and the injection system support portion 330 may be formed separately (such as illustrated), or the injection system base 310 may be formed as a single, monolithic element defining a connector portion 320 and a support portion 330. The material delivery device 340 is configured to deliver to a patient (e.g., inject into a patient) the contents of the chambers 202a, 202b of the multi-chamber device 210, injected by the multi-chamber device 210 via the injection system base 310 to the material delivery device 340. In the examples of embodiments described herein, the contents of the chambers 202a, 202b of the multi-chamber device 210 are injectable materials injected into a patient via a material delivery device 340 in the form of a needle. As such, the example of an embodiment of a material delivery device 340 illustrated in FIG. 5, FIG. 6A, and FIG. 6B extends from a proximal end 341 (supported by the injection system support portion 330) to a sharpened distal tip 340t at the distal end 343 of the material delivery device 340. It will be appreciated that in some embodiments, such as illustrated in FIG. 1, the length of the material delivery device 340 (from the proximal end 341 thereof to the distal end 343 thereof) may be longer than the length of the base 310. It will further be appreciated that the present disclosure need not be limited in this regard and other forms and configurations of injection systems and/or material delivery devices are within the scope and spirit of the present disclosure. To facilitate coupling of the multi-chamber device nozzles 222a, 222b and the injection system nozzle ports 322a, 322b with the material delivery device 340, the interior of the injection system connector portion 320 and injection system support portion 330 may be configured as/with a Y-connector 350, as may be seen with reference to the cross-sectional view in FIG. 6B, and the perspective view in FIG. 7. The injection system nozzle ports 322a, 322b are respectively fluidly coupled with the branches 352a, 352b of the Y-connector 350 which meet and flow into a common lumen 357 in fluid communication with the lumen 345 defined through the material delivery device 340. In the example of an embodiment illustrated in FIG. 6B and FIG. 7, the multi-chamber device nozzles 222a, 222b, the injection system nozzle ports 322a, 322b, and the branches 352a, 352b of the Y-connector 350 are offset from the longitudinal axis LA of the system, with one of the nozzles 222a, 222b, along with its respective port 322a, 322b, and respective branch 352a, 352b being closer to the longitudinal axis LA of the combining and/or delivery system 100 than the other. Such configuration may facilitate manufacture in some aspects. However, a more symmetrical arrangement/configuration is within the scope and spirit of the present disclosure, the present disclosure not being limited in this regard.

As described above, in embodiments of a combining and/or delivery system 100 with a multi-chamber device 210 (in contrast with a single chamber device), the components 204a, 204b within the chambers 202a, 202b, respectively, of the multi-chamber device 210 may remain separate until the time of delivery to a patient. In some embodiments, the components 204a, 204b react with each other and therefore are combined just prior to (e.g., immediately prior to) delivery to the patient so that the reaction begins or at least is completed within the patient. For instance, in embodiments in which the reaction between the components causes the combination of the components to form a material which is more solid (e.g., a gel-like material) than the separate components, it is desirable for the injectable material to be in a form which may pass through the injection system 300 and into the patient. Accordingly, in such embodiments, it is desirable for the reaction between the components of the injectable material (or at least the stage in which the combined components become more solidified) to take place once within the patient's body. In the example of an embodiment illustrated in FIG. 5 and FIG. 6B, combining of the contents of the first chamber 202a and the second chamber 202b of the multi-chamber device 210 occurs within the base 310 of the injection system 300. More particularly, in the example of an embodiment illustrated in FIG. 5 and FIG. 6B, combining (e.g., initial combining) of the contents of the first chamber 202a and the second chamber 202b of the multi-chamber device 210 occurs within the support portion 330 of the base 310 of the injection system 300. Even more particularly, a mixer 360 may be provided within a lumen 335 defined within the injection system support portion 330. The mixer 360 is configured to facilitate mixing of the contents of the first chamber 202a and the second chamber 202b of the multi-chamber device 210 as the plunger assembly 230 of the multi-chamber device 210 is advanced distally toward the distal end 213 of the multi-chamber device 210 to advance the plunger stoppers 232a, 232b distally through the chambers 202a, 202b to expel the components 204c, 204b out from the chambers 202a, 202b and into the lumen 335 defined within the base 310 of the injection system 300. In some embodiments, the mixer 360 has a plurality of radially-outwardly extending baffles 362, such as baffles known to those of ordinary skill in the art configured to facilitate mixing of the components 204c, 204b as the components are propelled distally through the injection system base 310 to the material delivery device 340. It will be appreciated that although reference is made to the precursor 204d, the above description is applicable to mixing of other components delivered by the multi-chamber device 210.

In view of the above, it will be appreciated that the sleeve lock 260 of the multi-chamber system 200 may be configured not just to couple the multi-chamber device 210 with the injection system 300, but also to couple the multi-chamber device 210 with an optional vial adaptor 250 and additional chamber 204c formed in a separate device 240. Similarly, the injection system 300 may be configured not just to be coupled with the multi-chamber device 210, but may also be configured to be coupled with one or more other material-containing devices. For instance, as described above, in some instances, it may be desirable to administer a pre-treatment material to a patient before delivering an injectable material from the multi-chamber device 210 to the patient. In accordance with various principles of the present disclosure, the sleeve-lock-engaging elements 324 of the injection system base 310 may be configured not only to engage the sleeve lock 260 carried by the multi-chamber device 210, but also to engage other sleeve locks. In some aspects, a pre-treatment may be delivered by a pre-treatment system having a single fluid outlet (referenced herein as a nozzle for the sake of convenience and without intent to limit) configured for ejecting material into a patient via the injection system 300. In such instances, a combining and/or delivery system 100 formed in accordance with various principles of the present disclosure may include an adaptor system 400 configured to adapt an injection system 300 with two ports 322a, 322b to be able to fluidly communicate with a pre-treatment system with a single nozzle. It will be appreciated that although the additional material-containing device to be coupled with an injection system 300 formed in accordance with various principles of the present disclosure is described herein as a pretreatment device delivering a pretreatment material to a patient via the injection system 300, the present disclosure need not be limited in this regard. Reference herein is made to a pretreatment device 500 simply for the sake of convenience, and without intent to limit.

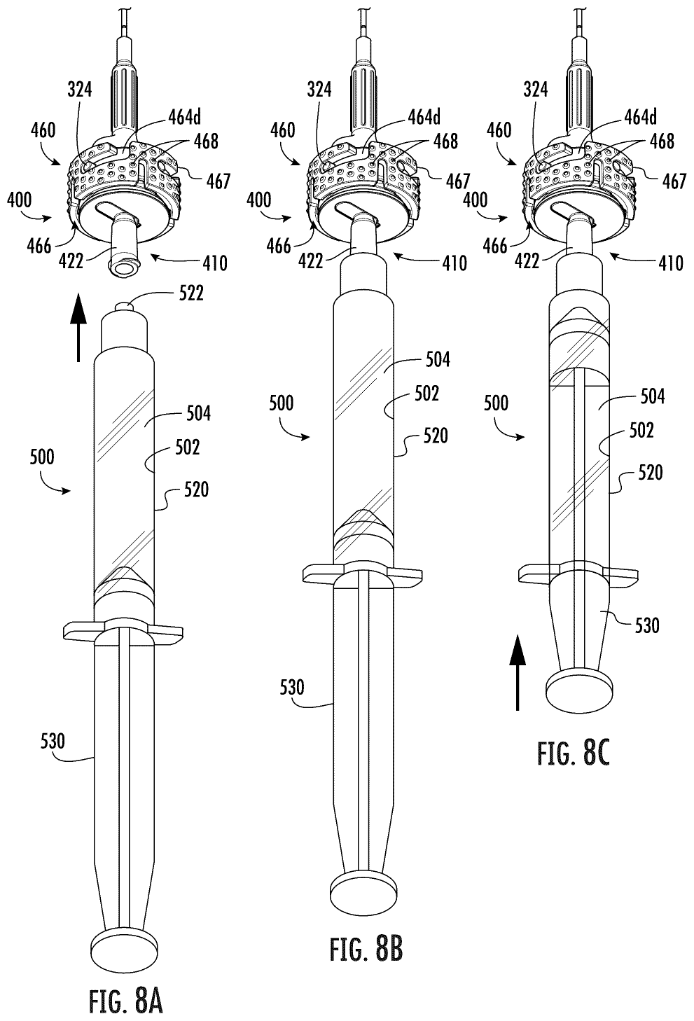

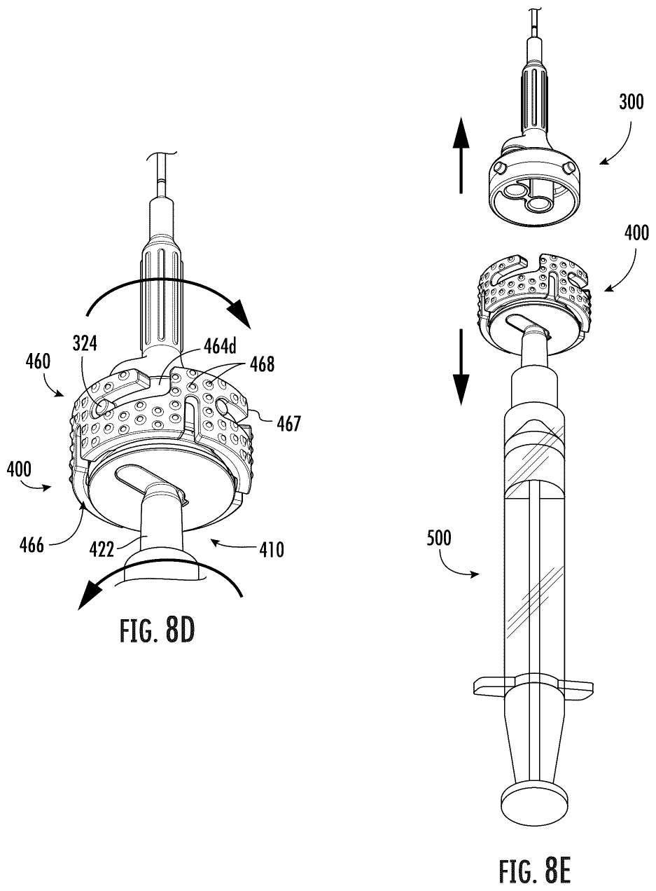

An example of an embodiment of an adaptor system 400 configured to facilitate coupling of an optional single-outlet pretreatment device 500 with an injection system 300 configured for coupling with a multi-chamber device 210 of the above-described combining and/or delivery system 100 is illustrated in FIG. 1 and FIG. 7. The illustrated example of an embodiment of an adaptor system 400 includes an adaptor 410 with a proximal end 411 defining an inlet portion 420 having a single port 422, and a distal end 413 defining an outlet portion 430 having a pair of nozzles 432a, 432b extending distally therefrom. It will be appreciated that the inlet portion 420 and the outlet portion 430 of an adaptor-system adaptor 410 formed in accordance with various principles of the present disclosure may be formed separately (such as illustrated), or as a single, monolithic element, the present disclosure not being limited in this regard. The single port 422 of the adaptor system 400 is configured to be fluidly coupled with a pretreatment device 500 having a barrel 520 with a single nozzle 522, such as illustrated in FIG. 8A-FIG. 8C. The pair of nozzles 432a, 432b are configured to be fluidly coupled, respectively, with the injection system nozzle ports 322a, 322b, as may be appreciated with reference to FIG. 7. The adaptor-system adaptor 410 optionally includes seals 416a, 416b (e.g., O-rings) positioned, respectively, between the nozzles 432a, 432b and the nozzle ports 322a, 322b, such as to seal against leakage.

The adaptor system 400 further includes a sleeve lock 460 configured to facilitate coupling of the adaptor-system adaptor 410 of the adaptor system 400 with the injection system 300. In the illustrated example of an embodiment, the adaptor-system sleeve lock 460 has one or more (e.g., 1-4, or more) distal engaging elements 464d configured to be coupled with the injection system sleeve-lock-engaging elements 324. Such engagement may be similar to the engagement between the injection system 300 and the sleeve lock 260 of the combining and/or delivery system 100 so that the injection system 300 may utilize the same sleeve-lock-engaging elements 224 for both sleeve locks 260, and 460. In the example of an embodiment illustrated in FIG. 7 and FIG. 8A-FIG. 8C, the adaptor-system sleeve lock 460 optionally is configured to be mounted with respect to the adapter system adaptor 410, such as to be carried on the adapter system adaptor 410, such as in a manner described above with respect to the sleeve lock 260 and multi-chamber device 210. In the example of an embodiment illustrated in FIG. 7, the adaptor system sleeve lock 460 includes flex slots 462 facilitating mounting of the adaptor-system sleeve lock 460 on the adaptor system adaptor 410, such as by an interference fit. The adaptor-system sleeve lock 460 may be rotatably mounted on the adaptor-system adaptor 410 with the assistance of one or more (e.g., 1-4, or more) proximal engaging elements 464p engaging one or more (e.g., 1-4, or more) sleeve-lock-engaging elements 434 defined on the adaptor-system adaptor 410 portion of the adaptor system 400. As may be appreciated, the flex slots 462 may facilitate engagement of the proximal engaging elements 464p over the sleeve-lock-engaging elements 434. Moreover, the adaptor-system sleeve lock 460 may be mounted and carried on the adaptor-system adaptor 410, such as similar to the manner in which the sleeve lock 260 is mounted and carried on the multi-chamber device 210 of the combining and/or delivery system 100.

The adaptor-system sleeve lock 460 may have a configuration similar to or substantially the same as the configuration of the multi-chamber system sleeve lock 260, such as to facilitate manufacture, ready coupling with the injection system 300, case of use, etc. For instance, the distal engaging elements 464d of the adaptor-system sleeve lock 460 may be configured as slots similar to the sleeve-lock-engaging elements 224 of the multi-chamber system sleeve lock 260. Additionally or alternatively, the proximal engaging elements 464p and sleeve-lock-engaging elements 434 of the adaptor system 400 may be configured similar to or substantially identical to corresponding elements of the multi-chamber system 200. For instance, the proximal engaging elements 464p of the adaptor-system sleeve lock 460 may be configured as one or more (e.g., 1-4, or more) ribs extending radially-inwardly from the inner surface 465 of the sleeve lock wall 466 for engagement with sleeve-lock-engaging elements 434 in the form of a circumferential groove around the outlet portion 430 of the adaptor-system adaptor 410. Such configuration allows for rotatable mounting of the adaptor-system sleeve lock 460 with respect to the adaptor-system adaptor 410, such as described above The adaptor system 400 may thus be coupled with the injection system 300 by axial and rotational movements similar to those described above with respect to the sleeve lock 260, with injection system 300 and the adaptor-system sleeve lock 460 being moved together in a generally axially direction to bring the sleeve-lock-engaging elements 324 of the injection system 300 and the distal engaging elements 464d of the adaptor-system sleeve lock 460 into initial engagement. The adaptor-system sleeve lock 460 and the injection system 300 may then be rotated with respect to each other (and optionally with respect to the adaptor-system adaptor 410) to interlock the sleeve-lock-engaging elements 324 and the distal engaging elements 464d so that the adaptor system 400 may not be readily withdrawn from the injection system 300. Optionally, the seals 416a, 416b provide a biasing force to hold the adaptor-system sleeve lock 460 and the injection system 300 in engagement to resist inadvertent rotation therebetween in a manner known to those of ordinary skill in the art.

Once the adaptor system 400 has been coupled with the injection system 300, the nozzle 522 of the pretreatment device 500 may be aligned with the port 422 of the adaptor system 400, as illustrated in FIG. 8A. The pretreatment device nozzle 522 may then be moved into engagement with the adaptor-system port 422, as illustrated in FIG. 8B, to fluidly couple the pretreatment device 500 with the injection system 300 with the assistance of the adaptor system 400. The plunger 530 of the pretreatment device 500 may then be advanced into the chamber 502 of the pretreatment device 500, as illustrated in FIG. 8C, to expel pretreatment material 504 out from the chamber 502 for delivery to a patient via the adaptor system 400 and the injection system 300.

The adaptor system 400 may be separated from the injection system 300 (e.g., after delivery of pretreatment material 504) by reversing the movements performed to couple the pretreatment device 500 and the injection system 300 For instance, the adaptor-system sleeve lock 460 and at least the injection system 300 may be rotated with respect to each other to move the injection system's sleeve-lock-engaging elements 224 to move to a position in which the injection system 300 and the adaptor system 400 may be moved generally axially apart to be separated/disengaged from each other, such as figuratively illustrated in FIG. 8D and FIG. 8E. It will be appreciated that the pretreatment device 500 may be separated/disengaged from the adaptor system 400 before the adaptor system 400 is separated/disengaged from the injection system 300. Optionally, the pretreatment device 500 may remain coupled with the adaptor system 400 to be separated/disengaged from the injection system 300 together with the adaptor system 400 (e.g., separated from the injection system 300 at the same time the adaptor system 400 is separated from the injection system 300). The injection system 300 may be left in place (with the material delivery device 340 inserted in the patient) for coupling with a multi-chamber device 210 such as described above, such as with the assistance of sleeve lock 260 such as described above.

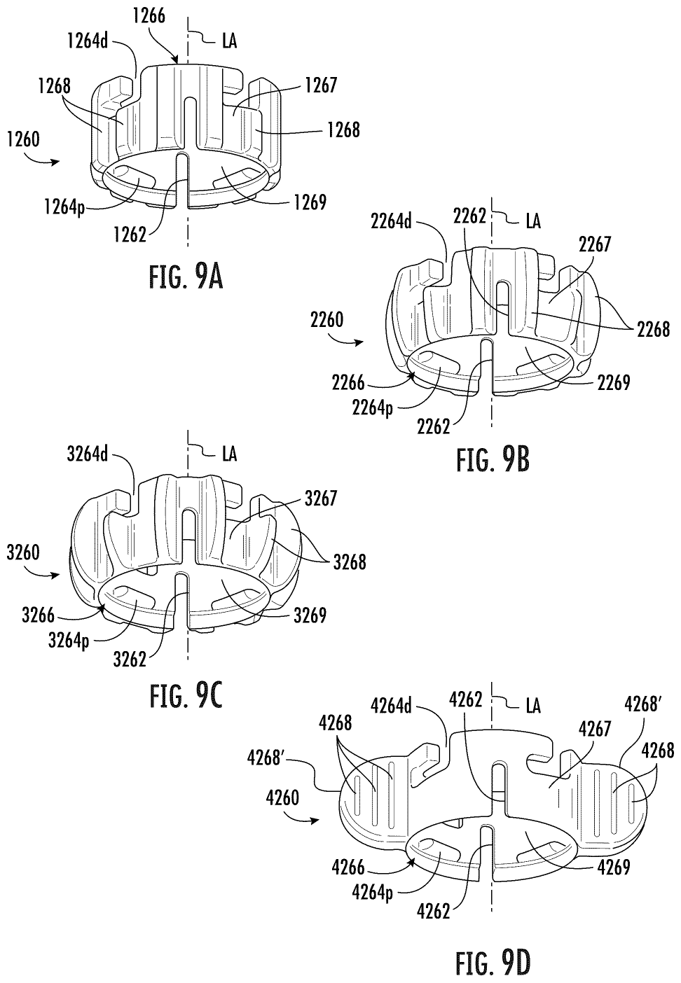

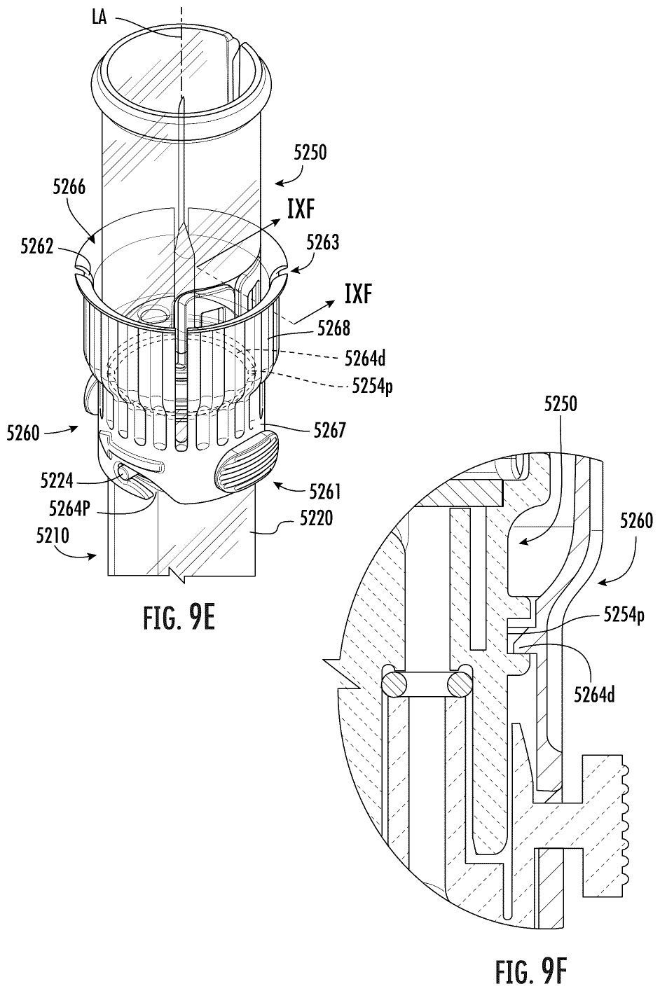

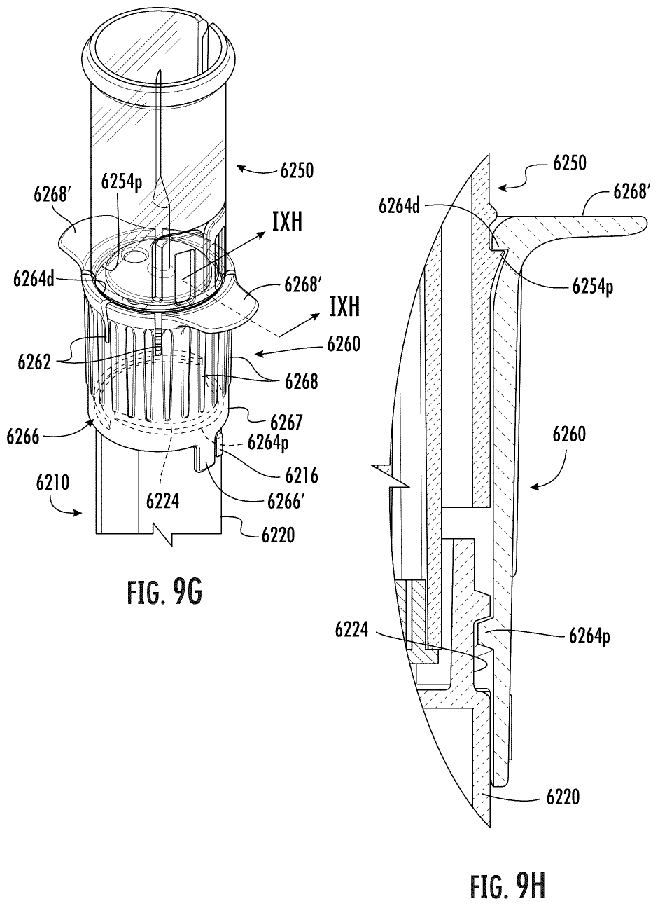

In accordance with various principles of the present disclosure, surface features may be provided on a sleeve lock formed in accordance with various principles of the present disclosure to facilitate grasping and optional rotation of the sleeve lock for coupling or decoupling with an element, component, device, system, etc., of a combining and/or delivery system 100 formed in accordance with various principles of the present disclosure, such as in a manner as described above. As may be appreciated with reference to the example of an embodiment of a sleeve lock 260 illustrated in FIG. 1, FIG. 2, FIG. 3A-FIG. 3H, surface features 268 which facilitate grasping of the sleeve lock 260 during relative rotation of the sleeve lock 260 and the vial adaptor 250 and/or relative rotation of the sleeve lock 260 and the injection system 300 may be in the form of a plurality of projections. The projections may be a series of projections, such as raised bumps, dimples, radially-outwardly extending from the exterior surface 267 of the sleeve lock wall 266, and optionally spaced apart from one another. The adaptor-system sleeve lock 460 illustrated in FIG. 7 and FIG. 8A-FIG. 8C may have similar or different surface features 468 on (e.g., extending radially-outwardly from) the exterior surface 467 of the wall 466 of the adaptor-system sleeve lock 460. Various other configurations of surface features may be provided on a sleeve lock formed in accordance with various principles of the present disclosure, and/or alternate configurations of the sleeve lock may further facilitate grasping and rotation thereof. For example, instead of projecting radially-outwardly, the surface features may be radially-inwardly extending, such as grooves. Additionally or alternatively, the surface features may be substantially contiguous (in contrast with being spaced apart, such as a wavy/sinusoidal gripping surface) and/or spacing among the individual surface features may be varied across the exterior surface of the sleeve lock wall. Additionally or alternatively, the location of the surface features may vary along the exterior surface of the sleeve lock wall. Various configurations of surface features known to those of ordinary skill in the art may be used instead of or in addition to those described herein. Various examples of embodiments of variations of surface features and/or configurations of sleeve locks formed in accordance with various principles of the present disclosure are illustrated in FIG. 9A-FIG. 9F. It will be appreciated that the examples of embodiments of sleeve locks illustrated in FIG. 9A-FIG. 9F may be used either with the multi-chamber system 200 or with the adaptor system 400. Accordingly, for the sake of convenience, and without intent to limit, descriptions of alternative configurations of sleeve locks are with reference to a generic sleeve lock, without reference to a system with which the sleeve lock is to be used.

In the examples of embodiments of sleeve locks illustrated in FIG. 9A, FIG. 9B, and FIG. 9C, instead of protrusions which are separated axially and circumferentially with respect to one another (e.g., bumps), the protrusions may be simply circumferentially separated from one another. For instance, the example of an embodiment of a sleeve lock 1260 illustrated in FIG. 9A has surface features 1268 configured as a plurality of circumferentially spaced apart ribs extending generally axially (generally along the longitudinal axis LA) and radially outwardly from the exterior surface 1267 of the wall 1266 of the sleeve lock 1260. The example of an embodiment of a sleeve lock 2260 illustrated in FIG. 9B has surface features 2268 configured as a plurality of circumferentially spaced apart, generally axially and radially-outwardly extending ribs 2268 similar to the ribs 1268 illustrated in FIG. 9A. However, whereas the ribs 1268 illustrated in FIG. 9A may have exterior surfaces generally parallel to the longitudinal axis LA of the sleeve lock 1260, the ribs 2268 illustrated in FIG. 9B have exterior gripping surfaces which are contoured (e.g., convex) relative to the longitudinal axis LA, such as to enhance grip thereof when handling and administering torque thereon during use; for added comfort, etc. Optionally, the length of the sleeve lock 3260 may be reduced and/or the curvature of the gripping surfaces of the surface features 3268 and/or extent away from the exterior surface 3267 of the sleeve lock wall 3266 increased. It will be appreciated that other variations to shapes, dimensions, distribution, and/or configurations of surface features improving grasping of a sleeve lock are within the scope and spirit of the present disclosure, the present disclosure not being limited by the illustrated examples of embodiments. Moreover, it will be appreciated that the examples of embodiments of sleeve locks 1260, 2260, 3260 may have other features in common with the example of an embodiment of a sleeve lock 260 described above and illustrated in FIG. 1, FIG. 2, and FIG. 3A-FIG. 3H, with common features being designated by reference numbers differing by multiples of 1000, and reference being made to the above descriptions of such features for the sake of brevity.

It will be appreciated that sleeve locks formed in accordance with various principles of the present disclosure may have gripping features other than surface features distributed along the exterior surface of the wall thereof. For instance, an example of an embodiment of a sleeve lock 4260 illustrated in FIG. 9D includes grips 4268′ in the form of flanges, wings, extensions, tabs, etc., extending radially-outwardly from an exterior surface 4267 of the sleeve lock wall 4266. Such grips 4268′ may include surface features 4268 in the form of protrusions or ribs, such as described above, to enhance gripping thereof. It will be appreciated that the example of an embodiment of a sleeve lock 4260 illustrated in FIG. 9D may have other features in common with the example of an embodiment of a sleeve lock 260 described above and illustrated in FIG. 1, FIG. 2, and FIG. 3A-FIG. 3H, with common features being designated by reference numbers differing by 4000, and reference being made to the above descriptions of such features for the sake of brevity.

Instead of a sleeve lock being mounted on a multi-chamber device and removably coupled with and decoupled from a vial adaptor of a multi-chamber system such as described above, a sleeve lock may have a reverse configuration, such as previously noted. For instance, a sleeve lock formed in accordance with various principles of the present disclosure may be mounted on a device other than a multi-chamber device, and configured to facilitate coupling/engaging and/or decoupling/disengaging with respect to a multi-chamber device. More particularly, the sleeve lock 5260 illustrated in FIG. 9E has a distal end 5263 configured to remain mounted on a vial adaptor 5250 and a proximal end 5261 configured for more ready engaging/coupling with and/or disengaging/decoupling from a multi-chamber device 5210. The sleeve lock 5260 thus is configured to be carried on the vial adaptor 5250 for more ready engagement/coupling and/or disengagement/decoupling with respect to a multi-chamber device 5210. For instance, the distal engaging elements 5264d of the sleeve lock 5260 may be in the form of one or more (e.g., 1-4, or more) radially-inwardly extending ribs (e.g., similar to the proximal engaging elements 264p of the sleeve lock 260 described above and illustrated, for example, in FIG. 2) configured to engage a corresponding groove 5254p in a vial adaptor 5250 (e.g., similar to the groove extending partially or fully around the barrel 220 of the multi-chamber device 210 described above and illustrated, for example, in FIG. 2), such as illustrated in FIG. 9F, showing a partial cross-sectional view along line IXF-IXF of FIG. 9E. Optionally, flex slots 5262 are defined through the sleeve lock wall 5266 to facilitate engagement of the distal engaging elements 5264d with the corresponding sleeve-lock-engaging elements 5254p of a vial adaptor. Likewise, the proximal engaging elements 5264p of the sleeve lock 5260 illustrated in FIG. 9E may be configured similar to the distal engaging elements 264d of the above-described sleeve lock 260, but to facilitate coupling with and decoupling from a multi-chamber device 5210. More particularly, the proximal engaging elements 5264p of the sleeve lock 5260 illustrated in FIG. 9E may be in the form of slots receiving and interlocking with one or more (e.g., 1-4, or more) sleeve-lock-engaging elements 5224 projecting radially-outwardly from a multi-chamber device 5210, such as described above with respect to the slots and corresponding projections of the example of an embodiment of a sleeve lock 260 and multi-chamber device 210 illustrated, for example, in FIG. 2. In some aspects, the example of an embodiment of a sleeve lock 5260 illustrated in FIG. 9E may have a greater length to extend along a longitudinal axis LA (e.g., of the sleeve lock 5260, as illustrated in FIG. 9E) than the above-described sleeve locks 260, 460, 1260, 2260, 3260, 4260. Such increase in length may be helpful in further stabilizing a vial adaptor 5250 with respect to the sleeve lock 5260.

To facilitate rotation of the sleeve lock 5260 with respect to the multi-chamber device 5210 (to couple the sleeve lock 5260 and associated vial adaptor 5250 with the multi-chamber device 5210, or to decouple the sleeve lock 5260 and associated vial adaptor 5250 from the multi-chamber device 5210), surface features 5268 may be defined along the exterior surface 5267 of the sleeve lock wall 5266. In the example of an embodiment illustrated in FIG. 9E, the surface features 5268 are in the form of a plurality of spaced apart generally axially extending grooves in the exterior surface 5267 of the sleeve lock wall 5266. However, it will be appreciated that the surface features 5268 may be in any of a variety of other configurations, such as any of the surface features described above. It will further be appreciated that the example of an embodiment of a sleeve lock 5260 illustrated in FIG. 9E may have other features in common with the example of an embodiment of a sleeve lock 260 described above and illustrated in FIG. 1, FIG. 2, and FIG. 3A-FIG. 3H, with common features being designated by reference numbers differing by 5000, and reference being made to the above descriptions of such features for the sake of brevity.

Various features of the above-described sleeve locks may be modified, and/or mixed and matched, and/or combined with further alternate features to result in further embodiments of sleeve locks in accordance with various principles of the present disclosure. For instance, the example of an embodiment of a sleeve lock 6260 illustrated in FIG. 9G may include various features which may be at least somewhat similar to features of the above-described sleeve locks 260, 460, 1260, 2260, 3260, 4260, 5260, such as surface features on the exterior surface 6267 of the sleeve lock wall 6266 configured to facilitate grasping and/or rotation of the sleeve lock 6260; grips 6268′ which extend radially-outwardly from the sleeve lock wall 6266; flex slots 6262 defined through the sleeve lock wall 6266 to facilitate mounting or removal of the sleeve lock 6260 with respect to a multi-chamber device and/or a vial adaptor; and various engaging-elements configured to engage and mount the sleeve lock 6260 with a multi-chamber device and/or vial adaptor. However, such features may be modified from the similar features of the above-described sleeve locks 260, 460, 1260, 2260, 3260, 4260, 5260 in one or more manners. For instance, whereas the sleeve lock 6260 may be elongated, like the example of an embodiment of a sleeve lock 5260 illustrated in FIG. 9E, instead of surface features in the form of elongated grooves, the surface features 6268 of the sleeve lock 6260 illustrated in FIG. 9G may be in the form of axially spaced apart, generally-longitudinally and radially-outwardly extending ribs.

Additionally or alternatively, although the grips 4268′, 6268′ of the examples of embodiments of sleeve locks 4260, 6260 illustrated in FIG. 9D and FIG. 9G, respectively, are in the form of radially-outwardly extending flanges, wings, extensions, tabs, etc., the orientations of the major planes in which these grips 4268′, 6268′ extend (i.e., the plane in which the majority of the surface area of the grips 4268′, 6268′ extends) are different. More particularly, the grips 4268′ of the example of an embodiment of a sleeve lock 4260 illustrated in FIG. 9D extend substantially vertically (e.g., parallel to or within about 45° from parallel to the longitudinal axis LA), such as to facilitate rotating the sleeve lock 4260 about the longitudinal axis LA. In contrast, the grips 6268′ of the example of an embodiment of a sleeve lock 6260 illustrated in FIG. 9F are substantially horizontal (e.g., perpendicular to or within about 45° from perpendicular to the longitudinal axis LA) to facilitate movement of the grips 6268′ in a direction generally along the longitudinal axis LA. Such movement of the grips 6268′ may cause a portion of the sleeve lock wall 6266 adjacent the grips 6268′ (e.g., positioned generally longitudinally along the grips 6268′) and between a pair of flex slots 6262 to flex with respect to portions of the sleeve lock wall 6266 on opposite sides of the pair of flex slots 6262. Furthermore, the flex slots 6262 of the above-described sleeve locks 260, 460, 1260, 2260, 3260, 4260, are positioned and configured to facilitate engagement of the sleeve lock with a multi-chamber device, whereas the flex slots 6262 of the sleeve lock 6260 illustrated in FIG. 9G are positioned and configured to facilitate engagement of the sleeve lock 6260 with a vial adaptor 6250.

Finally, whereas the above-described sleeve locks 260, 460, 1260, 2260, 3260, 4260, 5260 have one or more engaging elements to mount the sleeve lock with respect to a first device, and one or more engaging elements facilitating coupling and decoupling with respect to another device by generally axial as well as rotational relative movements therebetween, the one or more engaging elements 6264 of the example of an embodiment of a sleeve lock 6260 illustrated in FIG. 9G which are not configured for mounting the sleeve lock with respect to a first device are configured to facilitate simple rotational coupling and decoupling with respect to another device. For instance, as illustrated in phantom in FIG. 9G, and as may be appreciated with reference to the cross-sectional view along line IXH-IXH of FIG. 9G, as illustrated in FIG. 9H, the proximal engaging elements 6264p and the distal engaging elements 6264d are both generally circumferentially-extending engaging elements 6264p, 6264d configured to engage with corresponding generally circumferentially extending sleeve-lock-engaging elements of a multi-chamber device 6210 and a vial adaptor 6250 to rotatably mount the sleeve lock 6260 on both a barrel 6220 of the multi-chamber device 6210 as well as the vial adaptor 6250. More particularly, as illustrated in FIG. 9G, both the proximal engaging elements 6264p, and the distal engaging elements 6264d of the sleeve lock 6260 are configured as radially-outwardly generally circumferentially extending ribs which engage with the corresponding generally circumferentially extending ribs or grooves defined in the multi-chamber device 6210 and the vial adaptor 6250. And, like the example of an embodiment of a sleeve lock 5260 illustrated in FIG. 9E, distal engaging elements 6264d of the example of an embodiment of a sleeve lock 6260 illustrated in FIG. 9G and FIG. 9H are configured to mounted on a device other than a multi-chamber device. However, whereas the distal engaging elements 6264d extend circumferentially around the sleeve lock 6260, the proximal engaging elements 6264p extend helically around the sleeve lock 6260. And, whereas the proximal engaging elements 5264p of the example of an embodiment of a sleeve lock 5260 illustrated in FIG. 9E are engaged with engaging elements of another device (e.g., a multi-chamber device) by generally axial as well as rotational relative movements, the proximal engaging elements 6264p of the example of an embodiment of a sleeve lock 6260 illustrated in FIG. 9G are engaged with engaging elements of another device (e.g., a multi-chamber device) predominantly, if not almost completely by relative rotational movements therebetween. As such, whereas engagement of the distal engaging elements 6264d of the sleeve lock 6260 with engaging elements of a first device may be via an interference fit (e.g., snap fit), engagement of the proximal engaging elements 6264p of the sleeve lock 6260 with another device may be accomplished by as few as one full turn (360°), or even less than a full turn with respect to the other device (such as with Luer locks, as known by those of ordinary skill in the art). And, whereas the interference fit between the distal engaging elements 6264d of the sleeve lock 6260 and the engaging elements of the first device must be overcome to disengage the sleeve lock 6260 from the first device, disengagement of the proximal engaging elements 6264p of the sleeve lock 6260 from the engaging elements of the other device may be accomplished simply by relative rotation of the sleeve lock 6260 and the other device by as few as one full turn (360°), or even less than a full turn (such as with Luer locks, as known by those of ordinary skill in the art). As may be appreciated, the above-described grips 6268′ of the example of an embodiment of a sleeve lock 6260 illustrated in FIG. 9G may be pressed downwardly to flex a portion of the sleeve lock wall 6266 radially outwardly so that the distal engaging element 6264d may be disengaged from the sleeve-lock-engaging element 6254p of the vial adaptor 6250 to separate the vial adaptor 6250 from the multi-chamber device 6210. And, as may be appreciated with reference to FIG. 9G, proximal extensions 6266′ of the wall 6266 of the sleeve lock 6260 may limit relative rotation of the sleeve lock 6260 and the multi-chamber device 6210, such as upon engagement with a limit stop 6216 on the multi-chamber device 6210 (e.g., in the form of a radially-outwardly extending protrusion, such as on the barrel 6210 thereof).

Although the sleeve locks 5260, 6260 illustrated in FIG. 9E and FIG. 9G, respectively, are illustrated as configured for alignment and coupling with vial adaptors, it will be appreciated that these sleeve locks 5260, 6260 may be configured for coupling with an injection system such as described herein. Furthermore, it will be appreciated that various features of the sleeve locks 260, 460, 1260, 2260, 3260, 4260, 5260, 6260 illustrated in FIG. 9A, FIG. 9B, FIG. 9C, FIG. 9D, FIG. 9E, and FIG. 9G, respectively, have been described with reference to the sleeve lock 260 described above as used with the multi-chamber system 200, such features may be applied to a sleeve lock 460 configured for use with an adaptor system 400 such as described above.

In view of the above, it will be appreciated that, in contrast with prior systems having multiple components which need to be delivered, assembled with respect to one another, separated from one another, manipulated together with and separately from one another, etc., the present disclosure simplifies devices, systems, and methods for combining and/or delivering an injectable material to a patient and/or reduces (if not eliminates) human error occurring with the use of complex prior systems.

Further in view of the above, it should be understood that the various embodiments illustrated in the figures have several separate and independent features, which each, at least alone, has unique benefits which are desirable for, yet not critical to, the presently disclosed devices, systems, and methods. Therefore, the various separate features described herein need not all be present in order to achieve at least some of the desired characteristics and/or benefits described herein. For instance, only one of the various features described above may be present in a device or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An injectable material combining and/or delivery system comprising:
a multi-chamber device defining a first chamber and a second chamber therein;
a vial adaptor defining a vial-receiving chamber;
an injection system having a material delivery device configured to be fluidly coupled with said multi-chamber device to deliver, to a patient, a first component ejected from the first chamber into the injection system, and a second component ejected from the second chamber into the injection system; and
a sleeve lock configured to facilitate engagement and disengagement of said vial adaptor and said multi-chamber device as well as engagement and disengagement of said multi-chamber device and said injection system;
wherein:
said sleeve lock is mounted on said multi-chamber device; and
either of said vial adaptor or said injection system is axially and rotatably moved with respect to said sleeve lock to be engaged therewith or disengaged therefrom.

2. The system of claim 1, wherein said sleeve lock remains mounted on said multi-chamber device upon disengagement of said vial adaptor from said sleeve lock.

3. The system of claim 1, wherein said sleeve lock is rotatably mounted on said multi-chamber device.

4. The system of claim 1, further comprising a fluid exchange device within said vial adaptor.

5. The system of claim 4, wherein said vial adaptor and said fluid exchange device are configured to facilitate alignment of a vial within said vial adaptor to establish fluid communication between the vial and the first chamber of said multi-chamber device.

6. The system of claim 5, further comprising a vial defining a third chamber therein and having a two-part cap comprising an outer cap and a stopper configured to seal a component within the third chamber.

7. The system of claim 6, wherein:
said fluid exchange device comprises a needle and a fluid exchange spike supporting said needle and defining a fluid exchange lumen therethrough; and
said stopper of said two-part vial cap includes a wall section with reduced thickness to facilitate piercing therethrough by said needle of said fluid exchange device to facilitate passage of said fluid exchange spike through said stopper thereafter to fluidly communicate the first chamber and the third chamber.

8. The system of claim 6, wherein said vial adaptor includes vial-engaging elements configured to engage a portion of said outer cap of said vial.

9. The system of claim 1, wherein:
said multi-chamber device has a first nozzle in fluid communication with the first chamber and a second nozzle in fluid communication with the second chamber;
said injection system defines a first nozzle port configured to be fluidly coupled with said first nozzle, a second nozzle port configured to be fluidly coupled with said second nozzle, and a Y-connector fluidly coupling the first nozzle port and the second nozzle port with the material delivery device.

10. The system of claim 9, further comprising a mixer within said injection system configured to mix a first component ejected into the injection system from the first chamber of the multi-chamber device with a second component ejected into the injection system from the first chamber of the multi-chamber device.

11. The system of claim 9, further comprising an adaptor system having a sleeve lock configured to facilitate engagement and disengagement of said adaptor system and said injection system, said adaptor system having a single port configured to be fluidly coupled with a single-nozzle device, a first nozzle configured to be fluidly coupled with the first nozzle port of said injection system, and a second nozzle configured to be fluidly coupled with the second nozzle port of said injection system.

12. A multi-chamber system for combining components of an injectable material, said system comprising:
a multi-chamber device defining a first chamber and a second chamber therein;
a vial adaptor defining a vial-receiving chamber;
a sleeve lock configured to facilitate engagement and disengagement of said vial adaptor and said multi-chamber device; and
a fluid exchange device within said vial adaptor configured to fluidly communicate the first chamber of said multi-chamber device with a third chamber defined by a vial receivable within the vial-receiving chamber of said vial adaptor;
wherein:
said sleeve lock is mounted on said multi-chamber device; and
said vial adaptor is axially and rotatably moved with respect to said sleeve lock to be engaged therewith or disengaged therefrom.

13. The system of claim 12, further comprising a protective cap removably positioned within the vial-receiving chamber of said vial adaptor to shield said fluid exchange device.

14. The system of claim 12, wherein said vial adaptor includes radially-inwardly directed vial-engaging elements configured to engage a portion of a vial cap.

15. The system of claim 12, wherein:
said fluid exchange device comprises a needle and a fluid exchange spike supporting said needle and defining a fluid exchange lumen therethrough;
said multi-chamber device has a first nozzle in fluid communication with the first chamber; and
the fluid exchange lumen of said fluid exchange spike is in fluid communication with the first chamber of said multi-chamber device via a port defined in said vial adaptor in fluid communication with said first nozzle of said multi-chamber device.

16. A method for combining and/or delivering an injectable material to a patient, said method comprising:
coupling a vial adaptor with a multi-chamber device with the assistance of a sleeve lock engaging the vial adaptor and the multi-chamber device, the vial adaptor defining a vial-receiving chamber, and the multi-chamber device having a first chamber containing a first component and a second chamber containing a second component;
inserting a vial within the vial-receiving chamber of the vial adaptor, the vial defining a third chamber containing a third component;
fluidly communicating the first chamber within the multi-chamber device with the third chamber within the vial;
ejecting the first component out from the first chamber and into the third chamber;
aspirating the first component and the third component into the first chamber;
disengaging the vial adaptor, with the vial within the vial-receiving chamber, from the sleeve lock with the sleeve lock remaining engaged with the multi-chamber device;
coupling the multi-chamber device with an injection system with the assistance of the sleeve lock carried by the multi-chamber device;
ejecting the first and third component out from the first chamber and into the injection system;
ejecting the second component out from the second chamber and into the injection system; and
delivering the combined first component, second component, and third component to a patient via the injection system.

17. The method of claim 16, further comprising mixing the first component and the third component inside the third chamber.

18. The method of claim 16, further comprising mixing the second component with the combined first and third components within the injection system.

19. The method of claim 16, further comprising shaking the vial with the first component and the third component therein to combine the first component with the third component.

20. The method of claim 16, wherein coupling the sleeve lock with either the vial adaptor or the injection system comprising bringing the sleeve lock axially into engagement with one of the vial adaptor or the injection system, and then rotating the sleeve lock with respect to the one of the vial adaptor or the injection system to maintain axial engagement therebetween.
